SEQUENCE LISTING (6 sequences)

SEQ ID NO: 1 - RNA, 21 nt, synthetic construct
  source 1..21: mol_type = other RNA
ggatctacctgtcactacttt

SEQ ID NO: 2 - RNA, 21 nt, synthetic construct
  source 1..21: mol_type = other RNA
agtagtgacaggtagatccag

SEQ ID NO: 3 - RNA, 21 nt, synthetic construct
  source 1..21: mol_type = other RNA
cccagatggatctgcatttat

SEQ ID NO: 4 - RNA, 21 nt, synthetic construct
  source 1..21: mol_type = other RNA
cgacatccaccgctccattaa

SEQ ID NO: 5 - RNA, 19 nt, synthetic construct
  source 1..19: mol_type = other RNA
gtgctatgcctgtaacaag

SEQ ID NO: 6 - RNA, 19 nt, synthetic construct
  source 1..19: mol_type = other RNA
gacgaagcagaggtaatct

(19) United States
(12) Patent Application Publication
(10) Pub. No.: US 2025/0075210 A1
(43) Pub. Date: Mar. 6, 2025

(54) TARGETING THE YTHDF1/ARHGEF2 AXIS FOR CANCER TREATMENT

(71) Applicant: UNIVERSITY HEALTH NETWORK, Toronto (CA)

(72) Inventors: Shiyan WANG, Toronto (CA); Gang ZHENG, Toronto (CA); Housheng HE, Toronto (CA); Yulin MO, Toronto (CA)

(21) Appl. No.: 18/727,889

(22) PCT Filed: Jan. 12, 2023
(86) PCT No.: PCT/CA2023/050034

§ 371 Date: Jul. 10, 2024

Related U.S. Application Data
(60) Provisional application No. 63/299,054, filed on Jan. 13, 2022.

(51) Int. Cl. C12N 15/113; A61K 9/51; A61P 35/00

(52) U.S. Cl. CPC C12N 15/113; A61K 9/5123; A61P 35/00; C12N 2310/141

(57) ABSTRACT

There is described herein methods for the treatment of cancer in a subject comprising downregulating YTHDF1 or ARHGEF2 and also compounds and compositions for achieving the same.

### RELATED APPLICATION

This application claims priority to U.S. Provisional Application No. 63/299,054 filed on Jan. 13, 2022, the entire contents of which are incorporated herein by reference.

### FIELD OF THE INVENTION

The invention relates to cancer treatment that targets YTHDF1-ARHGEF2 axis and includes compositions that targeting the same.

### BACKGROUND OF THE INVENTION

Colorectal cancer (CRC) is one of the most common causes of cancer-related deaths worldwide.¹ CRC incidence and death rates have been increasing in younger adults.¹ Metastasis is the major cause of cancer death. In 50-60% of CRC patients, CRC eventually develops metastatic disease, most of which are unresectable liver and lung metastases.^{2,3} Metastatic CRC are treated with chemotherapy, targeted therapy and immunotherapy.² Chemotherapy and targeted therapy have demonstrated limited efficacy in the control of CRC metastasis. Metastatic CRCs with microsatellite instability-high/mismatch repair-deficient (MSI-H/dMMR) benefit from anti-PD1/PD-L1 immunotherapy. However, it is ineffective in the vast majority of CRCs that are microsatellite stable or mismatch repair-proficient (MSS/pMMR). Hence, there is an urgent need to identify novel druggable targets and prediction markers for CRC.

Over the past decade, intensive efforts such as The Cancer Genome Atlas (TCGA) and International Cancer Genome Consortium (ICGC) have led to the comprehensive genetic, epigenetic and transcriptomic characterization of CRC.^{4,5} In contrast to the well-established roles of these molecular determinants, post-transcriptional mechanisms that contribute to tumorigenesis remain poorly understood. Epitranscriptomics, which refers to post-transcriptional modifications of RNA transcriptome, plays critical regulatory roles in gene expression.⁶ Recent advances in sequencing technologies enabled examination of RNA modifications on a genome-wide scale,^{7,8} revealing significant dysregulation of RNA modifications during tumorigenesis.⁹

RNA N6-methyladenosine (m⁶A) is the most prevalent modification in eukaryotic mRNAs.¹⁰ m⁶A is dynamically regulated by the action of m⁶A writers (METTL3/METTL14/WTAP protein complex) and m⁶A erasers (FTO and ALKBH5). m⁶A modification in turn mediates the recruitment of m⁶A readers that associate m⁶A-modified RNAs to mRNA processing enzymes, influencing RNA export, splicing, translation and degradation.¹⁰ Three classes of m⁶A readers have been characterized, based on the manner by which they bind to m⁶A modified RNAs. 11 One major class is the YT521-B homology (YTH) domain family of proteins that directly bind to m⁶A modified sites (YTHDF1/2/3 and YTHDFC1/2).¹⁰ YTHDF1 has been identified as translation-facilitating m⁶A reader that recruits translation machinery to its target mRNAs in the cytoplasm.¹² Recent studies revealed that YTHDF1 plays crucial roles in various physiological processes such as learning and memory, CD8⁺ T cell priming and axon guidance.¹³⁻¹⁵ However, it remains to reveal whether and how m⁶A modifications translate pro-tumorigenic signals via YTHDF1.

### SUMMARY OF THE INVENTION

Applicant sought to address the consequence of m⁶A modifications in CRC mediated by YTHDF1. Applicant demonstrated that YTHDF1 is overexpressed in CRC, and its expression is associated with CRC metastasis. Integrative multiomic analysis highlighted a novel oncogenic epitranscriptome axis YTHDF1-m⁶A-ARHGEF2. The tumorigenic functions of this axis were validated in CRC cell lines, 3D organoid culture and Ythdf1 transgenic mice. Moreover, ARHGEF2 siRNA drug encapsulated by lipid nanoparticles (LNP) was developed for in vivo tumor treatment. Taken together, our work suggests YTHDF1-m⁶A-ARHGEF2 as a novel therapeutic target for CRC.

Accordingly, in an aspect, there is provided a method for the treatment of cancer in a subject in need thereof, the method comprising downregulating YTHDF1 or ARHGEF2.

In a further aspect, there is provided a nucleic acid molecule capable of selectively inhibiting, at least partially, YTHDF1 or ARHGEF2 expression.

In a further aspect, there is provided a pharmaceutical composition comprising the nucleic acid molecule described herein along with a pharmaceutically acceptable carrier.

In a further aspect, there is provided a use of the nucleic acid molecule described herein, in the preparation of a medicament for the treatment of cancer in a subject in need thereof.

### BRIEF DESCRIPTION OF FIGURES

These and other features of the preferred embodiments of the invention will become more apparent in the following detailed description in which reference is made to the appended drawings wherein:

FIG. 1. YTHDF1 is up-regulated by gene amplification in CRC. (A) mRNA abundances of main m⁶A regulators in the TCGA CRC cohort. Background shading indicates P-value; size and color of dot indicate the log₂ transformed fold change (log₂FC) of tumor over matched adjacent non-tumor. (B) mRNA expression of YTHDF1 in 151 paired CRC tissues in an internal cohort (paired t-test). (C) Representative images of YTHDF1 protein expression in CRC tumor and adjacent non-tumor tissues by IHC (upper). The protein levels of YTHDF1 in 53 matched CRC tumors and adjacent non-tumor tissues were assessed using Allred score (lower; paired t-test). Size of dot indicates the number of cases. Line thickness is proportional to the number of cases. (D) YTHDF1 mRNA expression and CNVs in 88 patients with advanced stage CRC from an internal cohort (left). Correlation between YTHDF1 mRNA expression and CNVs (right). (E) Top gene set depleted in CRC tumors with YTHDF1 copy number gain/amplification versus diploid in the TCGA CRC cohort.

FIG. 2. YTHDF1 promotes CRC cell growth and tumorigenesis in vivo. (A) Schematic representation of wildtype (WT) and mutant YTHDF1 protein in WT and Ythdf1^{Δ/Δ} mice. (B) Confirmation of genotype of WT and Ythdf1^{Δ/Δ} mice using reverse transcription PCR (RT-PCR) of DNA extracted from mouse tail (upper). Confirmation of YTHDF1 protein expression in mouse colon tissues from WT and Ythdf1^{Δ/Δ} mice by Western blot (lower). (C) Experimental design of AOM/DSS model. (D) Representative photographs of the colon (left), the colon tumor number (middle) and tumor burden (right) from WT and Ythdf1^{Δ/Δ} mice at the experimental endpoint in the AOM/DSS model. (E) Representative Hematoxylin and Eosin (H&E) staining of colon tumors from WT and Ythdf1^{Δ/Δ} mice. (F) Representative Ki-67 IHC of colon tumors from WT and Ythdf1^{Δ/Δ} mice (upper). Percentage of tumor cells with Ki-67-positive nuclear immunostaining in WT and Ythdf144 mice (lower). (G) Western blot analysis of protein expression of YTHDF1 upon YTHDF1 knockdown (upper) and overexpression (lower) in CRC cell lines. (H) Cell growth curve analysis of YTHDF1-knockdown (upper),-overexpressing (lower) and control cells by counting the number of cells at indicated time points. (I) Cell proliferation analysis of YTHDF1-knockdown (left),-overexpressing (right) and control cells by assessing BrdU incorporation 3 days after seeding. Each group contained four replicates. (J) Western blot analysis of protein expression of YTHDF1 upon YTHDF1 knockdown in CRC organoids (left). Cell viability of YTHDF1-knockdown and control CRC organoids 7 days after seeding (middle). Representative bright-field images of YTHDF1-knockdown and control CRC organoids (right). OE, overexpression. All histogram data are presented as mean±SD. Compared with control/WT group, *P<0.05; **P<01; ***P<0.001; ****P<0001.

FIG. 3. YTHDF1 promotes CRC cell migration and invasion in vitro and metastasis in vivo. (A) Representative images of transwell migration and Matrigel invasion assays of HCT116 cells transduced with the indicated lentivirus. (B) The cell migration and invasion abilities of YTHDF1-knockdown (upper),-overexpressing (lower) and control cells. (C) YTHDF1 knockdown inhibited liver metastasis in tail-vein metastasis model. Representative images of the formalin-fixed NOD/SCID mouse livers (upper left) and H&E staining of liver tissues (lower) 4 weeks after tail-vein injection of 1×10⁶ YTHDF1-knockdown and control HCT116 cells. Quantification of the numbers of metastatic nodules on the surface of mouse livers (upper right). (D) YTHDF1 knockdown inhibited lung metastasis in tail-vein metastasis model. Representative images of the NOD/SCID mouse lungs (upper left) and H&E staining of lung tissues (lower) 4 weeks after tail-vein injection of 1×10⁶ YTHDF1-knockdown and control HCT116 cells. Quantification of the proportions of tumor metastases in mouse lungs based on H&E staining (upper right). (E) YTHDF1 overexpression promoted tumor metastasis in liver metastasis model by intrasplenic injection. Representative images of the nude mouse livers (upper left) and H&E staining of liver tissues (lower) 4 weeks after intrasplenic injection of 5×10⁵ YTHDF1-overexpressing and control HCT116 cells. Quantification of the numbers of metastatic nodules on the surface of mouse livers (upper right). (F) YTHDF1 overexpression promoted lung metastasis in tail-vein metastasis model. Representative images of the nude mouse lungs (upper left) and H&E staining of lung tissues (lower) 8 weeks after tail-vein injection of 5×10⁵ YTHDF1-overexpressing and the control HCT116 cells. Quantification of the numbers of metastatic nodules on the surface of mouse lungs (upper right). Representative IHC staining of YTHDF1 (upper left) and quantification of YTHDF1 protein expression using Allred score (upper right) in YTHDF1-knockdown (G),-overexpressing (H) and control liver metastases. Representative IHC staining of Ki-67 (lower left) and quantification of the percentages of tumor cells with Ki-67-positive nuclear immunostaining (lower right) in YTHDF1-knockdown (G),-overexpressing (H) and control liver metastases. OE, overexpression. All histogram data are presented as mean±SD. Compared with control group, *P<0.05; **P<01; ***P<001; ****P<0001.

FIG. 4. Multiomic analyses identify YTHDF1 targets involved in CRC. (A) RNA-seq differential expression analysis of YTHDF1-knockdown versus control HCT116 cells. Heatmap of differentially expressed genes (DEGs) upon YTHDF1 knockdown (left). Volcano plot of statistical significance and fold changes in RNA abundances upon knockdown of YTHDF1 (Right). FDR, false discovery rate. Up-regulated and down-regulated DEGs are labeled with red and blue colors, respectively. (B) Top KEGG pathways enriched and depleted in YTHDF1-knockdown versus control HCT116 cells (left). Representative KEGG gene sets depleted in YTHDF1-knockdown versus control HCT116 cells (right). (C) ‘RICKMAN Metastasis DN’ gene signature enriched in YTHDF1-knockdown versus control HCT116 cells. (D) Schematic diagram of the strategy for pinpointing key YTHDF1 targets in CRC. (E) Enriched top motifs identified based on the top 5,000 m⁶A peaks in YTHDF1-knockdown and control HCT116 cells (left). The density of m⁶A peaks in 3 non-overlapping transcript segments: 5′-UTR, coding sequence (CDS) and 3′-UTR (right). (F) Venn diagram illustrating the overlap between DEGs by RNA-seq, genes containing m⁶A peaks by m⁶A MeRIP-seq and YTHDF1-bound genes by YTHDF1 RIP-seq. (G) Top enriched pathways of YTHDF1 targets. (H) Overlap between genes detected by protein mass spectroscopy and YTHDF1 target genes (left). The changes of protein abundance of 385 overlapped genes in two shYTHDF1 groups compared with the control group (right). (I) Translation efficiency (TE) analysis of candidate genes with significant alterations in protein abundance after YTHDF1 knockdown. PmR ratio was derived from quantitative proteomics and RNA-seq. TE by Ribo-seq data was the ratio of ribosome-protected mRNA fragments to total INPUT mRNA. (J) Correlation of YTHDF1 protein abundance with ARHGEF2 protein (left) and mRNA (right) abundance in the CPTAC CRC database (Pearson's correlation). (K) Western blot analysis of 12 pairs of matched CRC tumor and adjacent non-tumor tissue samples (left). Correlation between relative ARHGEF2 and YTHDF1 protein abundances in 12 pairs of matched CRC tumor and adjacent non-tumor tissue samples (Pearson's correlation) (right). (L) Representative images of YTHDF1 and ARHGEF2 protein abundance in CRC tissue arrays by IHC (left). CRC tumor samples were categorized into three groups according to Allred score (Group L: low expression; Group M: moderate expression; Group H: high expression). Correlation between YTHDF1 and ARHGEF2 protein levels (Pearson Chi-Square) (right). (M) YTHDF1 RIP IP (light blue), m⁶A MeRIP IP (orange) and INPUT (light purple) signals near the 3′-UTR region of ARHGEF2 mRNA (left). Relative m⁶A levels near the stop codon of ARHGEF2 mRNA in YTHDF1-knockdown and control HCT116 cells by m⁶A MeRIP PCR (right). All histogram data are presented as mean±SD. Compared with shCtrl, NS, not significant.

FIG. 5. ARHGEF2 is a key target of YTHDF1 in CRC. (A) Protein levels of YTHDF1 and ARHGEF2 by Western blot (left) and mRNA levels of YTHDF1 and ARHGEF2 by RNA-seq (middle) and qPCR (right) upon YTHDF1 knockdown in HCT116 cells. RPKM, Reads Per Kilobase Million. (B) Western blot analysis of protein expression of YTHDF1 and ARHGEF2 upon siRNA- and sgRNA-mediated knockdown of YTHDF1 in HCT116 cells. (C) Western blot analysis of protein expression of YTHDF1 and ARHGEF2 upon YTHDF1 knockdown in HT-29, RKO, PDO828 and PDO816 cells. (D) Western blot analysis of protein levels of YTHDF1 and ARHGEF2 upon YTHDF1 overexpression in HCT116 cells. (E) Representative IHC staining of ARHGEF2 in colon tumors (left) and quantification of ARHGEF2 protein level using Allred score in normal colon tissues and colon tumors from WT and Ythdf1^{Δ/Δ} mice at the experimental endpoint in the AOM/DSS model. (F) Western blot analysis of protein expression of active RhoA in HCT116 cells transfected with the indicated siRNA or/and plasmids. (G) Quantification of ROCK activity in HCT116 cells transfected with the indicated siRNA and plasmids. (H) F-actin staining by Alexa Fluor Plus 555 phalloidin (upper) and IF staining of focal adhesion marker paxillin (lower) in YTHDF1-knockdown and control HCT116 cells. DAPI was used to visualize nuclei. (I) ‘BERENJENO Transformed by RhoA UP’ gene signature depleted in YTHDF1-knockdown versus control HCT116 cells. (J) Cell growth analysis (left) and cell invasion abilities (right) of HCT116 cells transfected with the indicated siRNA or/and plasmids. (K) F-actin staining in HCT116 cells transfected with the indicated siRNA or/and plasmids. OE, overexpression. All histogram data are presented as mean±SD. NS, not significant; *P<05; **P<01; ***P<0.001; ****P<0.0001.

FIG. 6. ARHGEF2 contributes to YTHDF1-induced tumorigenesis and metastasis in vivo. (A) Western blot analysis of protein expression of YTHDF1 and ARHGEF2 in HCT116 cells transduced with the indicated lentivirus before injection in mice. (B) A representative image of the tumor xenografts harvested 19 days after the subcutaneous injection of 1×10⁶ HCT116 cells transduced with the indicated lentivirus into the nude mouse (left). Growth curves of HCT116 xenografts transduced with the indicated lentivirus (middle) and final tumor weights at the experimental endpoint (right). (C) Representative images of the formalin-fixed NOD/SCID mouse lungs 4 weeks after intravenous injection of 1×10⁶ HCT116 cells transduced with the indicated lentivirus (left). Quantification of the numbers of metastatic nodules on the surface of mouse lungs (right). (D) The presence of lung metastases as determined by H&E staining. (E) The dispersity of LNP 2′-OMe-modified siRNAs. (F) Western blot analysis of protein expression of YTHDF1 and ARHGEF2 in HCT116 cells transfected with the indicated LNP 2′-OMe-modified siRNAs for 48 h. (G) A representative image of tumor xenografts harvested 7 days after treatment with LNP 2′-OMe-modified siRNAs (left). Growth curves of HCT116 xenografts treated with the indicated LNP 2′-OMe-modified siRNAs (middle). The body weight of the LNP siRNAs-treated NOD/SCID mice at the experimental endpoint (right). (H) Representative ARHGEF2 IHC (upper left), quantification of ARHGEF2 level (upper right), representative Ki-67 IHC (lower left) and quantification of the percentages of tumor cells with Ki-67-positive nuclear immunostaining (lower right) in LNP siRNAs-treated xenografts. (I) Experimental design of LNP siRNA treatment in mouse liver metastasis model. (J) Representative images of the NOD/SCID mouse livers harvested 16 days after treatment with LNP 2′-OMe-modified siRNAs. (K) Quantification of the liver weight, the proportion of tumor metastases in mouse livers, the spleen weight and the body weight of the LNP siRNAs-treated mice at the experimental endpoint in mouse liver metastasis model. (L) The presence of liver metastases as determined by H&E staining. (M) Representative ARHGEF2 IHC (upper left), quantification of ARHGEF2 expression (upper right), representative Ki-67 IHC (lower left) and quantification of the percentages of tumor cells with Ki-67-positive nuclear immunostaining (lower right) in LNP siRNAs-treated liver metastases. OE, overexpression. All histogram data are presented as mean±SD. NS, not significant; *P<0.05; **P<0.01; ***P<001; ****P<0.0001.

FIG. 7. YTHDF1 expression and GSEA analysis in TCGA database. (A) YTHDF1 mRNA expression in the TCGA Pan-Cancer cohort. Background shading indicates P-value; size and color of dot indicate the log₂ transformed fold change (log₂FC) of tumors over matched adjacent non-tumor. (B) mRNA levels of YTHDF1 in CRC tumors and paired adjacent non-tumor tissues in the TCGA CRC cohort. (C) CNVs of YTHDF1 and top CNV-enriched genes generated by GISTIC algorithms from the TCGA CRC cohort. (D) Correlation between YTHDF1 mRNA abundance and CNVs in the TCGA CRC cohort. (E) ‘RICKMAN Metastasis DN’ gene signature in CRC tumors with YTHDF1 high (higher than 50th percentile) versus low (lower than 50th percentile) mRNA abundance in the TCGA CRC cohort.

FIG. 8. Generation of Ythdf1^{Δ/Δ} mice. (A) Location of sgRNAs for CRISPR-mediated specific knockout are depicted. Primers KO-F/KO-R1/R2 were used to identify the deleted region in Ythdf1^{Δ/Δ} mice, generating a shorter PCR product. (B) Direct sequencing of KO-F/KO-R1/R2-amplified PCR products to confirm gene deletion in Ythdf1^{Δ/Δ} mice.

FIG. 9. Effects of YTHDF1 on cell migration, invasion and metastasis. (A) Representative images of transwell migration and Matrigel invasion assays of HT-29 cells transduced with the indicated lentivirus. (B) Western blot analysis of protein level of YTHDF1 upon YTHDF1 knockdown in RKO cells transduced with the indicated lentivirus before injection in mice. (C) Representative images of the NOD/SCID mouse livers (left) and quantification of the numbers of metastatic nodules on the surface of the NOD/SCID mouse livers (right) 4 weeks after tail-vein injection of 1×10⁶ YTHDF1-knockdown and control RKO cells. (D) Western blot analysis of protein level of YTHDF1 upon YTHDF1 overexpression in RKO cells transduced with the indicated lentivirus before injection in mice. (E) Representative images of the NOD/SCID mouse livers (left) and quantification of the numbers of metastatic nodules on the surface of the NOD/SCID mouse livers (right) 4 weeks after tail-vein injection of 5×10⁵ YTHDF1-knockdown and control RKO cells. OE, overexpression. All histogram data are presented as mean±SD. Compared with control group, **P<0.01; ****P<0.0001.

FIG. 10. Multiomics integration analysis. (A) Correlation of gene expression between control (left) and shYTHDF1 (right) groups. (B) Principal component analysis (PCA) plot of YTHDF1-knockdown and control HCT116 RNA-seq libraries after normalization. (C) Cellular localization of YTHDF1 in untreated and LMB-treated HCT116 cells. A nucleocytoplasmic shuttling protein DACT2 served as a positive control for nuclear retention after LMB treatment. Cells were transfected with Flag-tagged YTHDF1 or DACT2 construct followed by exposure to 50 nM LMB for 4 h. (D) m⁶A levels of shared 3,527 genes among the three groups (One-way ANOVA test; P=0.86). Shared genes were defined as genes with m⁶A peaks in all three groups. The maximum ratio of each peak's IP signal to the corresponding INPUT signal was used for calculating m⁶A level of each gene. (E) Correlation analysis of protein abundance changes between the two shYTHDF1 groups compared to control.

FIG. 11. Representative IHC staining of ARHGEF2 in normal colon tissues from WT and Ythdf1^{Δ/Δ} mice at the experimental endpoint in the AOM/DSS model.

FIG. 12. ARHGEF2 promotes CRC cell proliferation and invasion. (A) Western blot analysis of protein expression of ARHGEF2 upon ARHGEF2 knockdown in HCT116 cells. (B) Bright-field microscopy images of ARHGEF2-knockdown and control HCT116 cells. (C) Cell growth curve analysis of ARHGEF2-knockdown and control HCT116 cells. (D) Cell invasion abilities of ARHGEF2-knockdown and HCT116 control cells. All histogram data are presented as mean±SD. Compared with control group, **P<0.01; ***P<0.001; ****P<0.0001.

FIG. 13. Effects of ARHGEF2 knockdown in CRC cells. (A) Western blot analysis of ARHGEF2 protein level in CRC cell lines. (B) Western blot analysis of ARHGEF2 protein level upon ARHGEF2 knockdown in CRC cell lines. (C) Cell viability of ARHGEF2-knockdown and control CRC cells 2 days after seeding (left). The cell migration abilities of ARHGEF2-knockdown and control CRC cells (right). Each group contained three replicates. All histogram data are presented as mean±SD. Compared with control group, NS, not significant; *P<0.05; **P<0.01; ***P<0.001.

FIG. 14. LNP siRNA treatment in mouse liver metastasis model. (A) Representative images of the formalin-fixed NOD/SCID mouse livers. (B) Representative ARHGEF2 IHC in LNP siRNAs-treated liver metastases.

FIG. 15. Schematic of LNP siRNA treatment.

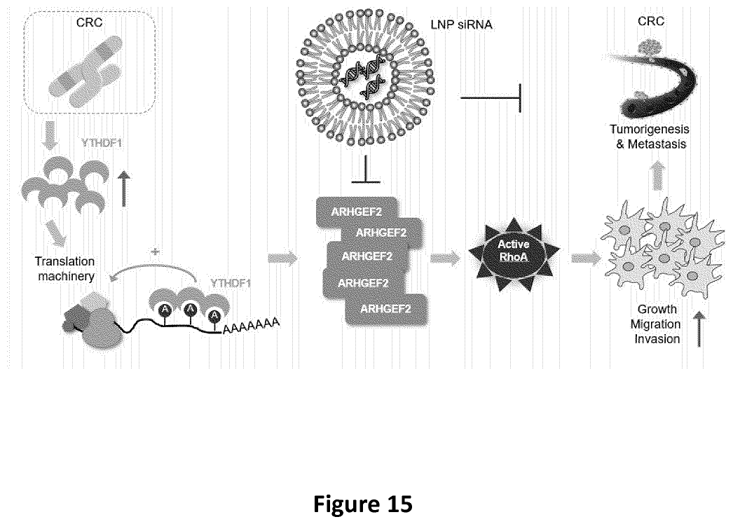

### DETAILED DESCRIPTION

In the following description, numerous specific details are set forth to provide a thorough understanding of the invention. However, it is understood that the invention may be practiced without these specific details.

N6-methyladenosine (m⁶A) governs the fate of RNAs through m⁶A readers. Colorectal cancer (CRC) exhibits aberrant m⁶A modifications and expression of m⁶A regulators. However, how m⁶A readers interpret oncogenic m⁶A methylome to promote malignant transformation remains to be illustrated.

Ythdf1 knockout mouse was generated to determine the effect of Ythdf1 in CRC tumorigenesis in vivo. Multiomic analysis of RNA-sequencing, m⁶A methylated RNA immunoprecipitation sequencing, YTHDF1 RNA immunoprecipitation sequencing and proteomics were performed to unravel targets of YTHDF1 in CRC. The therapeutic potential of targeting YTHDF1-m⁶A-ARHGEF2 was evaluated using siRNA encapsulated by lipid nanoparticles (LNP).

DNA copy number gain of YTHDF1 is a frequent event in CRC and contributes to its overexpression. High expression of YTHDF1 is significantly associated with metastatic gene signature in patient tumors. Ythdf1 knockout in mice dampened tumor growth in an inflammatory CRC model. YTHDF1 promotes cell growth in CRC cell lines and primary organoids, and lung and liver metastasis in vivo. Integrative multiomics analysis identified RhoA activator ARHGEF2 as a key downstream target of YTHDF1. YTHDF1 binds to m⁶A sites of ARHGEF2 mRNA, resulting in enhanced translation of ARHGEF2. Ectopic expression of ARHGEF2 restored impaired RhoA signaling, cell growth and metastatic ability both in vitro and in vivo caused by YTHDF1 loss, verifying that ARHGEF2 is a key target of YTHDF1. Finally, ARHGEF2 siRNA delivered by LNP significantly suppressed tumor growth and metastasis in vivo.

We identify a novel oncogenic epitranscriptome axis of YTHDF1-m⁶A-ARHGEF2, which regulates CRC tumorigenesis and metastasis. siRNA-delivering LNP drug validated the therapeutic potential of targeting this axis in CRC.

In an aspect, there is provided a method for the treatment of cancer in a subject in need thereof, the method comprising downregulating YTHDF1 or ARHGEF2.

The methods, uses, and compositions described herein include embodiments relating to agents capable of inhibiting, downregulating, or abolishing the activity and/or the expression of YTHDF1 or ARHGEF2, total or partially, or any combination of one or more such inhibitor agents. As long as the agent possesses the inhibitory function (e.g., inhibits YTHDF1 or ARHGEF2 expression and/or activity), the inhibitor agent may be selected from any class of compound. Thus, the inhibitors as used herein refer to any compound that reduces, inhibits, downregulates, or abolishes the expression and/or function of YTHDF1 or ARHGEF2, total or partially, or an agent suitable for neutralizing, reducing, or inhibiting the expression or function of YTHDF1 or ARHGEF2. The inhibitors described herein may exert action by any mechanism including, for example, by binding to YTHDF1 or ARHGEF2 transcripts.

In some embodiments, the method comprises downregulating the YTHDF1-ARHGEF2 axis.

In some embodiments, downregulating ARHGEF2 comprises administration of a nucleic acid molecule to the subject, the nucleic acid molecule capable of selectively inhibiting, at least partially, YTHDF1 or ARHGEF2 expression.

Examples of nucleic acid molecules include antisense oligonucleotides and in addition, those capable of mediating RNA interference which include a duplex RNA such as an SiRNA (small interfering RNA), miRNA (micro RNA), shRNA (short hairpin RNA), ddRNA (DNA-directed RNA), piRNA (Piwi-interacting RNA), or rasiRNA (repeat associated siRNA), and modified forms thereof.

In some embodiments, the nucleic acid molecule is a shRNA, siRNA, mRNA or antisense oligonucleotide targeted to ARHGEF2 or YTHDF1.

In some embodiments, the siRNA comprises sense strand GGAUCUACCUGUCACUACUtt (SEQ ID NO. 1) and antisense sense strand AGUAGUGACAGGUAGAUCCag (SEQ ID NO. 2).

In some embodiments, the shRNA comprises shYTHDF1-1: 5′-CCCAGATGGATCTGCATTTAT-3′ (SEQ ID NO. 3); shYTHDF1-2: 5′-CGACATCCACCGCTCCATTAA-3′ (SEQ ID NO. 4); shARHGEF2-1: 5′-GTGCTATGCCTGTAACAAG-3′ (SEQ ID NO. 5); or shARHGEF2-2: 5′-GACGAAGCAGAGGTAATCT-3′ (SEQ ID NO. 6).

The nucleic acid molecules and methods of this invention may be pooled, or used in combination to down regulate the expression of genes that encode ARHGEF2 or YTHDF1.

The term “oligonucleotide” as used herein refers to a nucleic acid molecule comprising from about 1 to about 100 nucleotides, more preferably from 1 to 80 nucleotides, and even more preferably from about 4 to about 35 nucleotides. This may include nucleic acid molecules of variable length that correspond either to the sense strand or to the non-coding strand of a target nucleic acid sequence.

“Antisense oligonucleotides” are complementary to a region of a target gene and are capable of hybridizing to the target gene sequence and inhibiting gene expression. Gene expression is inhibited through hybridization of an AON to a specific messenger RNA (mRNA) sense target according to the Watson-Crick base pairing, typically in which adenosine and thymidine (uracil in mRNA) or guanosine and cytidine interact through hydrogen bonding. Without being bound to any theory, two mechanisms are generally thought to account for these effects, the first being hybridization with impaired translation of targeted mRNA, the second being the induction of RNase H or similar enzymes with associated degradation of target mRNA.

Oligonucleotide compounds in accordance with the present invention also include siRNAs (small interfering RNAs) and the RISCs (RNA-induced silencing complexes) containing them that result from the RNAi (RNA interference) approach. The RNAi approach is a tool for the inhibition of target gene expression. RNAi is based on an ancient anti-viral defence mechanism in lower eukaryotes. It is induced by double-stranded RNA and its processing to typically 21-23 nt siRNAs, which cause the degradation of homologous endogenous mRNA after hybridizing to the target mRNA in a single stranded fashion with the assistance of the RISC complex. The way in which RNAi inhibits target gene expression remains to be fully elucidated, but presently, RNAi serves as an attractive choice approach to generate loss-of-function phenotypes across a broad spectrum of eukaryotic species, such as nematodes, flies, plants, fungi and mammals.

A short hairpin RNA or small hairpin RNA (shRNA/Hairpin Vector) is an artificial RNA molecule with a tight hairpin turn that can be used to silence target gene expression via RNA interference (RNAi). Expression of shRNA in cells is typically accomplished by delivery of plasmids or through viral or bacterial vectors.

Oligonucleotide compounds in accordance with the present invention also include microRNA (miRNA). “MicroRNA” are single-stranded RNA molecules, typically of about 21-23 nucleotides in length, which regulate gene expression in a hybridization dependent manner. Typically, miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, typically at the 3′end of the mRNA, and their main function is to downregulate gene expression.

In some embodiments, the nucleic acid molecule is administered to the subject in a lipid nanoparticle as delivery vehicle.

The composition and methods disclosed herein can also be used in treating various kinds of malignant tumors in a subject.

In some embodiments, the cancer is selected from the group consisting of colorectal adenocarcinoma, stomach adenocarcinoma, lung adenocarcinoma, breast carcinoma, cholangiocarcinoma, liver hepatocellular carcinoma, head/neck squamous cell carcinoma, uterine corpus endometrial carcinoma, high-risk Wilms tumor, esophageal carcinoma, bladder urothelial carcinoma, kidney renal papillary cell carcinoma, prostate adenocarcinoma, giolblastoma multiforme, cervical squamous cell carcinoma/endocervical adenocarcinoma, pheochromocytoma/paraganglioma, and pancreatic adenocarcinoma. Preferably, the cancer is colorectal adenocarcinoma.

In an aspect, there is provided a nucleic acid molecule capable of selectively inhibiting, at least partially, YTHDF1 or ARHGEF2 expression.

In some embodiments, the nucleic acid molecule is a siRNA. In other embodiments, the nucleic acid molecule is a miRNA or an antisense oligonucleotide.

In some embodiments, the nucleic acid molecule is encapsulated within a lipid nanoparticle.

In some embodiments, the nucleic acid molecule described herein is for use in the treatment of cancer in a subject in need thereof.

In an aspect, there is provided a use of the nucleic acid molecule described herein, in the preparation of a medicament for the treatment of cancer in a subject in need thereof.

In a further aspect, there is provided a pharmaceutical composition comprising the nucleic acid molecule described herein along with a pharmaceutically acceptable carrier.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

Abbreviations used herein include the following: 2′-OMe, 2′-O-Methyl; AOM, azoxymethane; BP, Biological Process; CRC, colorectal cancer; CRISPR, clustered regularly interspaced short palindromic repeats; CPTAC, Clinical Proteomic Tumor Analysis Consortium; DSS, dextran sulfate sodium; FFPE, formalin-fixed paraffin embedded; FDA, Food and Drug Administration; GSEA, Gene Set Enrichment Analysis; GO, Gene Ontology; GEF, guanine nucleotide exchange factor; IHC, immunohistochemistry; IF, immunofluorescence; iTRAQ, isobaric tagging for relative and absolute quantification; KEGG, Kyoto Encyclopedia of Genes and Genomes; LMB, Leptomycin B; LNP, lipid nanoparticles; m⁶A, N6-methyladenosine; MeRIP, methylated RNA immunoprecipitation; mRNA, messenger RNA; MSI, microsatellite instability; NMD, nonsense-mediated mRNA decay; PCR; quantitative polymerase chain reaction; PmR, protein-to-mRNA; PDI, Polydispersity Index; qPCR, quantitative PCR; Ribo-seq, ribosome profiling; RNA-seq, RNA-sequencing; RIP, RNA immunoprecipitation; ROCK, Rho-associated protein kinase; sgRNA, single-guide RNA; shRNA, short hairpin RNA; TCGA, The Cancer Genome Atlas; UTR, untranslated region; and WT, wildtype.

The advantages of the present invention are further illustrated by the following examples. The examples and their particular details set forth herein are presented for illustration only and should not be construed as a limitation on the claims of the present invention.

### EXAMPLES

### Methods and Materials

### Primary CRC and Adjacent Non-Tumor Tissue Samples

151 snap-frozen paired CRC tumors and adjacent non-tumor tissues for quantitative PCR (qPCR) analysis, 12 snap-frozen paired CRC tumors and adjacent non-tumor tissues for Western blot analysis and 22 formalin-fixed paraffin embedded (FFPE) tissue blocks from matched CRC and non-tumor mucosa were obtained from CRC patients who underwent surgery at Prince of Wales Hospital, The Chinese University of Hong Kong.¹⁶ Biopsy samples from primary CRC tumor and adjacent non-tumor were obtained from CRC patients at the time of operation before any therapeutic intervention. 31 FFPE tissue blocks from matched CRC and non-tumor mucosa were kindly provided by Dr. Xiaohong Wang from Peking University Cancer Hospital. Tissue microarray slides containing 208 CRC cases were kindly provided by Dr. Wei Kang from Department of Anatomical and Cellular Pathology, The Chinese University of Hong Kong. The study protocols have been approved by the Clinical Research Ethics Committee of Prince of Wales Hospital, The Chinese University of Hong Kong and Peking University Cancer Hospital. All patients provided written informed consent for obtaining the study specimens. This study was carried out in accordance with the Declaration of Helsinki of the World Medical Association.

### Cell Lines

The CRC cell lines HCT116 (Cat. #CCL-247), HT-29 (Cat. #HTB-38), RKO (Cat. #CRL-2577), DLD-1 (Cat. #CCL-221), LoVo (Cat. #CCL-229), LS 180 (Cat. #CL-187) and SW1116 (Cat. #CCL-233) were obtained from the American Type Culture Collection (ATCC). Cell lines were maintained according to protocols from ATCC. Human colon cancer patient-derived organoids PDO828 and PDO816 were kindly provided by Dr. Catherine Adell O'Brien in Princess Margaret Cancer Center in the University of Toronto. Organoids were cultured in Corning Growth Factor Reduced Matrigel matrix (Corning) in advanced DMEM/F12 medium (Thermo Fisher Scientific, Cat. #12634028) supplemented with 1% Penicillin-Streptomycin (Thermo Fisher Scientific, Cat. #15140148), HEPES (10 μM; Thermo Fisher Scientific), GlutaMAX™ Supplement (1:100; Thermo Fisher Scientific), serum free B-27™ Supplement (1:50; Thermo Fisher Scientific), 1.25 mM N-Acetyl-L-cysteine (MilliporeSigma), 10 nM [Leu¹⁵]-Gastrin I human (MilliporeSigma), 50 ng/mL recombinant murine epithelial growth factor (Thermo Fisher Scientific), 100 ng/mL murine Noggin (Peprotech) and 0.5 μM A 83-01 (Tocris Bioscience).^{m1}

Plasmids and siRNAs

The full-length Flag-tagged YTHDF1 cDNA (NM_017798.4) was amplified and cloned into the pCDNA3.1+ expression vector (Thermo Fisher Scientific) and lentiviral vector pLVX-Puro (Takara Bio USA). The full-length Flag-tagged ARHGEF2 cDNA (NM 001162383.2) was amplified and cloned into the pCDNA3.1+ expression vector (Thermo Fisher Scientific) and lentiviral vector pLV-Neo (Inovogen Tech. Co.). pCDNA3.1-Flag-DACT2 was constructed as described previously^{m2}. YTHDF1, ARHGEF2 and negative control (shCtrl) shRNAs were cloned into lentivirus shRNA expression plasmid pLVshRNA-puro (Inovogen Tech. Co.). The shRNA target sequences were as below: shCtrl: 5′-CCACATGAAGCAGCACGACTT-3′; shYTHDF1-1: 5′-CCCAGATGGATCTGCATTTAT-3′; shYTHDF1-2: 5′-CGACATCCACCGCTCCATTAA-3′; shARHGEF2-1: 5′-GTGCTATGCCTGTAACAAG-3′; shARHGEF2-2: 5′-GACGAAGCAGAGGTAATCT-3′). For siRNA knockdown, cells were transfected with YTHDF1 siRNAs (Silencer® Select; Thermo Fisher Scientific) (SiYTHDF1-1: Cat. #4392420-s29743; SiYTHDF1-2: Cat. #4392420-s29745), ARHGEF2 siRNAs (Silencer® Select; Thermo Fisher Scientific) (siARHGEF2-1: Cat. #4392420-s17545; siARHGEF2-2: Cat. #4392420-s17546) or control siRNA (siCtrl; Silencer™ Negative Control No. 1 siRNA; Cat. #AM4635; Thermo Fisher Scientific).

### Generation of Ythdf1^{Δ/Δ} Mice

Ythdf1 knockout mice were generated using the CRISPR/Cas9 system. Cas9 mRNA and two sgRNAs (sg1 and sg2) were microinjected into fertilized embryos of C57BL/6J mice. Deletion in exon 4 was confirmed by Sanger sequencing. Genotyping was performed by PCR of tail-snip DNA using genotyping primers (data not shown). All mice were housed in a Specific Pathogen Free (SPF) environment for the duration of the study. All experiments in this study were approved by the Institutional Animal Care and Use Committee at Xiamen University.

### IF and Stress Fibre Staining

Cells were fixed with 4% paraformaldehyde/PBS and permeabilized. Nonspecific binding sites were blocked with 1% BSA/PBS. Primary antibodies were applied to the cells at room temperature for 2 h. After PBS wash, secondary antibodies with desired fluorescence probes were applied. Then cells were washed and mounted with ProLong™ Gold Antifade Mountant with DAPI (4′,6-diamidino-2-phenylindole) (Thermo Fisher Scientific). To inhibit nuclear export, 24 h post transfection, Leptomycin B (LMB; MilliporeSigma; dissolved in methanol) was added to the culture medium to a final concentration of 50 nM for 4 h. To stain stress fibre, cells were fixed 16 h after seeding and stained with Alexa Fluor Plus 555 phalloidin solution (Cat. #A30106; Thermo Fisher Scientific; dissolved in DMSO) for 30 min according to manufacturer's instructions.

### Cell Viability Assay

The CellTiter-Blue Cell Viability Assay kit (Promega) was used to quantify the growth of organoids. For other CRC cells, the growth curves were evaluated by conventional hemocytometer counting chambers.

### BrdU Cell Proliferation Assay

˜5000 YTHDF1-knockdown,-overexpressing and the corresponding control HCT116 and HT-29 cells were seeded in 96-well plates. Three days after seeding, cell proliferation was assessed by BrdU Cell Proliferation Assay Kit (Cat. #6813; Cell Signaling Technology) following the manufacturer's instructions.

### Migration and Invasion Assay

Corning® BioCoat® Growth Factor Reduced Matrigel Invasion Chamber with 8.0 μm PET Membrane (Cat. #354483, Corning) and Corning® 6.5 mm Transwell® with 8.0 μm Pore Polycarbonate Membrane Insert, Sterile (Cat. #3422, Corning) were used to evaluate cell invasion and migration, respectively. CRC cells (YTHDF1 overexpression: 2×10⁴ cells per insert; other assays: 4×10⁴ cells per insert) were suspended in the culture medium containing 1% FBS and seeded into the upper chambers (invasion: Matrigel-coated; migration: uncoated). The culture medium containing 10% FBS was added into the lower chambers as a chemoattractant. After 24˜60 h incubation, cells on the lower surface were fixed and stained for counting.

### Xenograft Assays

For xenograft assay, ˜1×10⁶ HCT116 cells were subcutaneously injected into the right flanks of 4˜6 weeks old male nude or NOD/SCID mice. Body weight and tumor volume were measured every 2˜3 days until the endpoint.

### In Vivo Metastasis Assays

The mouse model of lung metastasis was established by tail-vein injection of HCT116 cells into the nude mouse or NOD/SCID mouse. Specifically, HCT116 cells (overexpression: 5×10⁵ cells per mouse; knockdown: 1×10⁶ cells per mouse) were injected intravenously through the tail vein into each 4-6 weeks old male mouse. The health status of the mice was monitored at least once a week at the beginning and daily when the humane intervention points were approaching. The humane intervention points were that the mice in one of the experimental groups were in poor health conditions, such as suppressed activity, being unresponsive to touch, marked hunched posture, dehydration, rough hair coat, dyspnea and weight loss over 20% of initial body weight. The nude mice and NOD/SCID mice were sacrificed 7˜8 and 5˜6 weeks after tail-vein injection, respectively. The lungs from each mouse were excised and embedded in paraffin. The lung metastasis was assessed either by the number of visible pulmonary metastatic nodules or the proportion of tumor metastases in the mouse lung when tumor nodules were indistinguishable.

The mouse model of liver metastasis was established by either tail-vein injection of HCT116 or RKO cells into the NOD/SCID mouse (overexpression: 5×10⁵ cells per mouse; knockdown: 1×10⁶ cells per mouse) or intrasplenic injection of HCT116 cells into the nude mouse or NOD/SCID mouse. In the intrasplenic injection model, enrofloxacin in the drinking water as a prophylactic oral antibiotic was administered to mice 72 h prior to surgery. Mice were anesthetized with Buprenorphine Sustained-Release (SR). ˜1 cm incision was made in the left upper abdominal wall and ˜1 cm incision was made in the peritoneum to expose the mouse spleen. Moistened sterile cotton swab was used to gently exteriorize the spleen. HCT116 cells (overexpression: 5×10⁵ cells per mouse; nanoparticle siRNA drug treatment: 0.75×10⁶ cells per mouse) were injected into each mouse with a 27 G needle. After the spleen was returned to the abdominal cavity, the muscle layer and skin were closed and subcutaneous fluid therapy was administered. The health status, abdominal distension and the size of tumor formed in the spleen were strictly monitored. The humane intervention points were that the mice in one of the experimental groups had marked abdominal distension, marked tumor in the spleen or in poor health conditions. The mice were sacrificed 3˜4 weeks after intrasplenic injection. The mouse liver was excised and fixed for histological examination. The liver metastasis was assessed either by the number of visible liver metastatic nodules or the liver weight and the proportion of tumor metastases in the mouse liver when tumor nodules were indistinguishable. All animal experiments were approved by the Animal Experimentation Ethics Committee of The Chinese University of Hong Kong and The University Health Network.

### IHC

Paraffin sections at 4 μm thickness were dried in a 60° C. oven for 2 h before staining. IHC was performed according to the manufacturer's guidelines using BenchMark XT Automated Slide Staining System (Ventana Medical Systems) with standard antigen retrieval (CC1, pH 8.0, Cat. #950-124, Ventana Medical Systems). The dilutions for YTHDF1 (Cat. #17479-1-AP, Proteintech) were 1:500 for human clinical samples, 1:500 for YTHDF1 knockdown assays and 1:600 for YTHDF1 overexpression assays in mouse metastases models, respectively. The dilution for ARHGEF2 (Cat. #ab155785, Abcam) was 1:500 for all samples. The dilutions for Ki-67 (Cat. #MA5-14520, Thermo Fisher Scientific) were 1:600 for YTHDF1 overexpression assay in mouse liver metastasis model and 1:500 for the rest of the samples. The primary antibodies were incubated for 32 min. Biotinylated anti-rabbit IgG antibody (Cat. #BA-1000-1.5, Vector Laboratories) was added to slides at 1:200 for 12 min. The primary-secondary complex was then visualized with Ventana iVIEW™ DAB Detection Kit (Cat. #760-091, Ventana Medical Systems). The slides were counterstained with Harris hematoxylin, dehydrated in graded alcohol, cleared in xylene and coverslipped in Permount. Sections were evaluated by two pathologists. The expression levels of YTHDF1 and ARHGEF2 were evaluated by the Allred scoring system. For the samples from mouse metastasis models, Allred score in each specific metastatic site from a given sample was evaluated (proportion score+intensity score). Average Allred score for a given sample from mouse metastasis models=sum of all (Allred score×area proportion (%)) in all metastatic sites). For the rest of samples, Allred score=proportion score+intensity score. For the tissue array, CRC tumor samples were categorized into three groups according to Allred score (Group L: Allred score 1˜5, low expression; Group M: Allred score 6, moderate expression; Group H: Allred score 7˜8, high expression).

### RNA-Seq

Total RNAs were purified with RNeasy Mini Kit (Cat. #74106; QIAGEN), and the DNA was digested by RNase-Free DNase Set (Cat. #79254; QIAGEN). RNA-seq libraries were constructed with TruSeq Stranded mRNA Library Prep kit (Cat. #20020595; Illumina) using 4 μg total RNA according to the manufacturer's protocol. Libraries were sequenced as 45 bp single-end reads in duplicates at ˜40 million reads per library using Illumina NextSeq 500 platform (Illumina).

m⁶A MeRIP-Seq and m⁶A MeRIP PCR

m⁶A MeRIP was performed according to the method we developed previously described.^{m3} Briefly, total RNA from cells in culture was extracted by TRIzol reagent (Cat. #15596018, Thermo Fisher Scientific) and treated with DNase I (Cat. #04716728001, Roche Diagnostics) according to the manufacturer's instructions. 9 ng of E. coli K-12 (Cat. #EC1, MilliporeSigma) total RNA was added to 4 μg of human total RNA sample before RNA fragmentation. For m⁶A MeRIP, 30 μl of protein A magnetic beads (Cat. #10002D, Thermo Fisher Scientific) and 30 μl of protein G magnetic beads (Cat. #10004D; Thermo Fisher Scientific) were washed by IP buffer (150 mM NaCl, 10 mM Tris-HCl [pH 7.5], 0.1% IGEPAL CA-630), resuspended in 500 μl of IP buffer containing 5 μg anti-m⁶A antibody (Cat. #ABE572, MilliporeSigma) at 4° C. overnight. The antibody-bead mixture was washed and resuspended in 500 μl of the IP reaction mixture containing 4 μg fragmented total RNA, 100 ul of 5×IP buffer, and 5 μl of RNasin Plus RNase Inhibitor (Cat. #N2611, Promega) and incubated for 2 hours at 4° C. The RNA reaction mixture was then washed using a low/high salt-washing method. After washing, the m⁶A-enriched fragmented RNA was purified using RNeasy Mini Kit (Cat. #74106, QIAGEN). m⁶A MeRIP INPUT and IP were subjected to library construction using SMARTer Stranded Total RNA-Seq Kit version 2-Pico Input Mammalian (Cat. #634413, Takara/Clontech) according to the manufacturer's protocol. A purified library mix was subjected for 45 bp single-end sequencing at ˜30 million reads per library using a NextSeq 500/550 High Output Kit v2.5 (75 Cycles) (Cat. #20024906, Illumina). m⁶A levels of the genes were determined by m⁶A MeRIP qPCR by assessing the percentage of a target gene in IP fraction relative to that in INPUT fraction: % INPUT=2\hat{ }(Ct of target gene in IP-adjusted Ct of target gene in INPUT).

### Proteomic Profiling

Briefly, the protein was extracted using a lysis buffer (4% SDS, 20 mM HEPES and 1×protease inhibitor cocktail). Protein concentrations were measured by Bio-Rad protein assay kit (Bio-Rad). 100 μg of protein was tryptic-digested to peptides following filter-aided sample preparation (FASP) protocol in a 30 KD filter unit (MilliporeSigma). The resulting peptides were collected and desalted by C18 ZipTip (MilliporeSigma). 50 μg of protein extract were labeled with iTRAQ® Reagent-8PLEX Multiplex Kit (MilliporeSigma) according to the manufacturer's protocols. Peptides were directly loaded on a self-pack C18 analytical reverse phase column (ID 75 μm×15 cm, 200 Å, 3 μm particles) at a flow rate of 300 nL/min and eluted by a 60 minu LC gradient of 8% to 22% acetonitrile (ACN) in 0.1% formic acid (FA). The eluted peptides were analyzed by Thermo Scientific Orbitrap Fusion Lumos Tribrid Mass Spectrometer machine in a data-dependent acquisition mode, setting as following: 1 microscan for MS1 scans at 120,000 resolution (FWHM at m/z 400), MS2 at 30,000 resolution (FWHM at m/z 400); Full MS mass range: m/z 300-2000; MS/MS mass range: m/z 100-2000. AGC target for MS2 is 50000, maximum injection time is 60 ms, HCD collision energy 35%. The MS raw data is searched against the UniProt Homo sapiens database by software Mascot Daemon (v2.5.1). Search parameters were set as following: trypsin as digestion enzyme and maximum miss cleavage is 2, Precursor Mass Tolerance 20 ppm, Fragment Mass Tolerance 0.02 Da, Carbamidomethyl on Cysteine as fix modification, FDR is set at 0.01. Three biological replicates were included for each group to increase the reliability of isobaric-tags for quantitation.

### YTHDF1 RIP-Seq

2×10⁷ HCT116 cells were harvested after 24 h transfection of Flag-tagged YTHDF1 plasmid. The protein was extracted with 1000 μl of lysis buffer (150 mM KCl, 10 mM HEPES pH 7.6, 2 mM EDTA, 0.5% IGEPAL CA-630, 0.5 mM DTT, 10 μl of RNasin Plus RNase Inhibitor per 1000 μl lysis buffer, 1× protease inhibitor cocktail). 50 μl cell lysate was saved as INPUT, mixed with 1 ml TRIzol. 30 μl of protein A magnetic beads and 30 μl of protein G magnetic beads were washed by NT2 buffer (200 mM NaCl, 50 mM HEPES pH 7.6, 2 mM EDTA, 0.05% IGEPAL CA-630, 0.5 mM DTT), resuspended in 500 μl of NT2 buffer containing 5 μg anti-Flag antibody (Cat. #F1804, MilliporeSigma) at 4° C. overnight. The antibody-bead mixture was washed and resuspended in 995 μl of cell lysate and incubated for 4 h at 4° C. The beads were then washed 8 times with 1 ml ice-cold NT2 buffer and re-suspended with 1 ml TRIzol as IP. RNAs from INPUT and IP were extracted and subjected to library construction using SMARTer Stranded Total RNA-Seq Kit version 2-Pico Input Mammalian (Takara/Clontech) according to the manufacturer's protocol. Libraries were sequenced as 45 bp single-end reads at ˜40 million reads per library using Illumina NextSeq 500 platform (Illumina).

### High Throughput Sequencing Data Alignment and Analysis

RNA-sequencing (RNA-seq), m⁶A methylated RNA immunoprecipitation sequencing (m⁶A MeRIP-seq) and YTHDF1 RNA immunoprecipitation sequencing (RIP-seq) reads were aligned to the human reference genome hg38 by using STAR (version 2.4.2a) ma with the reference annotation GENCODE version 25.^{m5} HTSeq/DESeq2 pipeline was then applied to quantify and normalize the IP and INPUT per each gene for each type of data.^{m6,m7} To identify the differential expressed genes (DEGs) from RNA-seq data, the DESeq2 was employed with the criteria that abs (log₂ (shYTHDF1/shControl))>0.58 and FDR<0.05. m⁶A MeRIP-seq uniquely mapped reads for every sample were subsampled to the same read depth (30 million), and reads without duplication were used for peak calling by MeTPeak^{m3}. For the identification of the m⁶A peak summit and motif discovery, in-house scripts and DREME (MEME Suite: version 4.11.2.1) were used.^{m8} The peak summit-based m⁶A distributions along mRNA transcripts were performed by Guitar (version 1.20.1).^{m2} For RIP-seq data, the genes bound by YTHDF1 were defined as genes with enrichment fold change (log₂ (IP/INPUT)) greater than 1.^{m10} Functional enrichment analysis was performed through the web server g: Profiler^{m11} using annotated genes as background. The R package clusterProfiler (version 3.10.1) was used for pre-ranked GSEA based on log₂ (RNA levels fold change)×−log₁₀ (p value).^{m12}

### RhoA Activation Assay

Active GTP-bound RhoA levels were measured using RhoA Pull-Down Activation Assay Biochem Kit (Cytoskeleton). Briefly, cell lysates were incubated with rhotekin-RBD beads for 1 h at 4° C. The protein/beads complexes were washed with the provided wash buffer and the bound proteins were eluted in 2X Bolt™ LDS Sample Buffer (Thermo Fisher Scientific) and then analyzed by Western blot.

### ROCK Activity Assay

ROCK activity was determined by 96-Well ROCK Activity Assay Kit (Cat. #STA-416, Cell Biolabs) following the manufacturer's instructions. Briefly, 8 μg of total protein was added in each well and incubated at 30° C. for 1 h. After adding Stop Solution, the absorbance at 450 nm was measured.

### ARHGEF2 Sensitivity Assay

CRC cells were transfected with ARHGEF2 siRNAs or control siRNA (Thermo Fisher Scientific). 24 h post transfection, the cells were seeded at ˜5000 cells per well in 96-well plates. Two days after seeding, cell viability was assessed by MTT assay (Cat. #M6494; Thermo Fisher Scientific) following the manufacturer's instructions. In addition, 48 h after siRNA transfection, the CRC cells were seeded into the upper chambers of Corning® 6.5 mm Transwell® with 8.0 μm Pore Polycarbonate Membrane Insert, Sterile (Cat. #3422, Corning) for analysis of cell migration.

Lipid Nanoparticle (LNP) Formulation of siRNA Drug

siRNA-loaded LNP formulations were formed using microfluidic rapid mixing method as previously reported.^{m13} 1,2-distearoyl-sn-glycero-3-phosphocholine (DSPC), cholesterol and 1,2-dimyristoyl-rac-glycero3-methoxy (poly(ethylene glycol))-2000 (DMG-PEG2000), were purchased from Avanti Polar Lipids, Inc (Alabaster, AL). DLin-MC3-DMA was purchased from Organix, Inc. (Woburn, MA). Lipids were mixed in ethanol at a molar ratio of DLin-MC3DMA/DSPC/Cholesterol/DMG-PEG2000: 50/10/38.5/1.5. 2′-O-Methyl (2′-OMe) modified siRNA was dissolved in a 25 mM acetate buffer (pH=4.0). The two phases were mixed through herringbone microfluidic chips (microfluidic ChipShop, Germany) at a volumetric flow rate ratio of 3:1 (aqueous to ethanol). The mixed solution was dialyzed against PBS overnight to remove the ethanol and change external pH to 7.4. Afterwards the formulations were passed through a 0.22 μm filter before use. The hydrodynamic size and dispersity of LNP was characterized by a Zetasizer Nano ZS (Malvern Instruments, United Kingdom). siRNA encapsulation efficiency was measured by Ribogreen Assay (Thermo Fisher Scientific).

### Data Availability

All sequencing data of this study have been deposited in NCBI's Gene Expression Omnibus (GEO) database under accession number GSE159425 (https://www.ncbi.nlm.nih.gov/geo/query/acc.cgi?acc=GSE159425; reviewer token: qjifuqesdbavtwn).

### Statistical Analysis

Data are presented as mean±standard deviation (SD). The independent Student's t test was used to compare the difference between 2 preselected groups. The chi-square test was used for comparison of incidence. The difference in growth rate between the two groups was determined by repeated measures analysis of variance (ANOVA). Value of P<0.05 was taken as statistical significance.

### Results and Discussion

YTHDF1 is Amplified in CRC and Associated with Metastatic Gene Signature

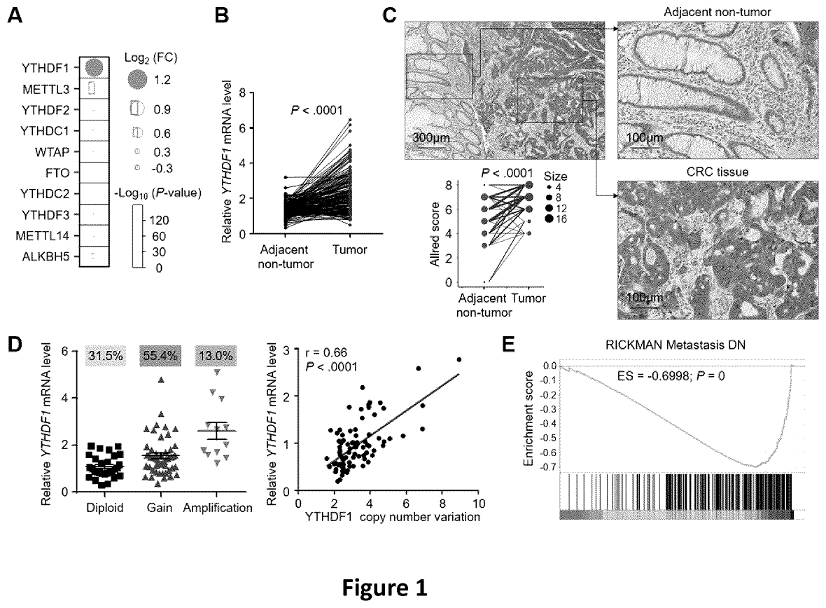

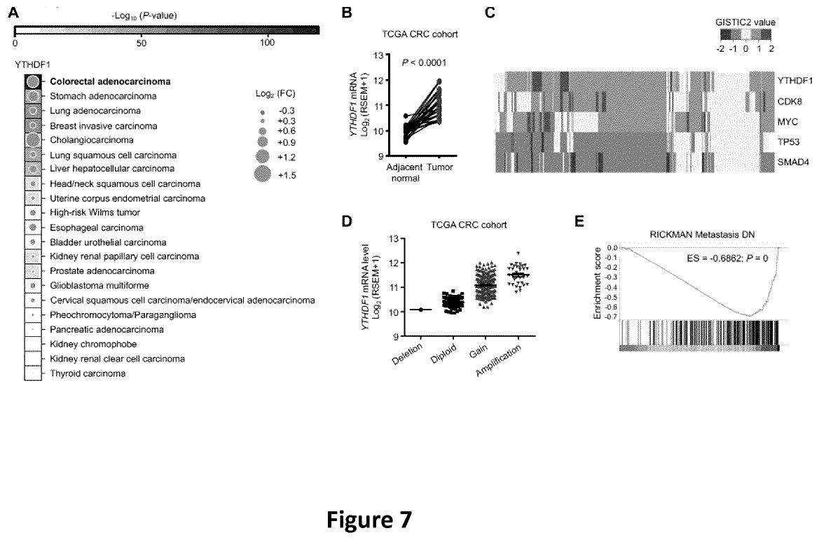

We first analyzed mRNA expression of m⁶A regulators in the TCGA CRC dataset, revealing that YTHDF1 is the most highly up-regulated one in CRC compared to adjacent non-tumor tissues (FIG. 1A). Pan-cancer analysis showed that up-regulation of YTHDF1 mRNA was most prominent in CRC (FIG. 7A). YTHDF1 mRNA level was also significantly increased in CRC tumor tissues compared with their matched adjacent non-tumor tissues in an internal CRC cohort (N=151; P<0.0001; FIG. 1B) and the TCGA cohort (N=30; P<0.0001; FIG. 7B). We next performed immunohistochemistry (IHC) and confirmed the overexpression of YTHDF1 protein in primary CRC (N=53; P<0.0001; FIG. 1C).

YTHDF1 is located in 20q13.31, a region that is frequently amplified in CRC. To determine if YTHDF1 is up-regulated via gene amplification in CRC, we first analyzed the YTHDF1 copy number variations (CNVs) in our CRC cohort (N=88; stage III and IV) using a specific TaqMan probe against YTHDF1, and found that YTHDF1 copy number gain (3˜4 copies) and gene amplification (>5 copies) were present in 55.4% (49/88) and 13% (11/88) of CRC tumors, respectively (FIG. 1D). GISTIC analysis of the TCGA CRC cohort demonstrated 68.3% (417/610) of CRC exhibited YTHDF1 copy number gain (GISTIC annotation of +1) while 7.9% (48/610) had gene amplification (GISTIC annotation of +2) (FIG. 71C).¹⁷ Moreover, integrative genomic analyses of copy number and mRNA revealed that YTHDF1 CNVs was positively correlated with its mRNA expression in our cohort (r=0.66; P<0.0001; FIG. 1D) and the TCGA CRC cohort (r=0.71; P<0.0001; FIG. 7D), indicating that copy number gain/amplification of YTHDF1 contributes to its up-regulation in CRC.

Amplification and upregulation of YTHDF1 in CRC compared to non-tumor tissues indicate its function in CRC tumorigenesis. We further compared the gene expression profiles of CRCs from the TCGA cohort with or without YTHDF1 gain/amplification using Gene Set Enrichment Analysis (GSEA) (data not shown). Among all the gene sets depleted in CRC tumors with YTHDF1 gain/amplification as compared to those YTHDF1 diploid tumors, the ‘RICKMAN Metastasis DN’ gene signature (genes down-regulated in metastatic versus non-metastatic head and neck squamous cell carcinoma) was the most enriched gene set (FIG. 1E). Consistently, this gene signature was significantly depleted in CRC tumors with high YTHDF1 mRNA expression (FIG. 7E). Moreover, high YTHDF1 mRNA level was significantly associated with lymph node status (P=0.02), metastasis (P=0.05) and advanced TNM stage (P=0.02) in the TCGA cohort (data not shown), indicating a potential function of YTHDF1 in regulating CRC metastasis.

### Deletion of Ythdf1 Reduces Tumorigenesis in an Inflammatory CRC Mouse Model

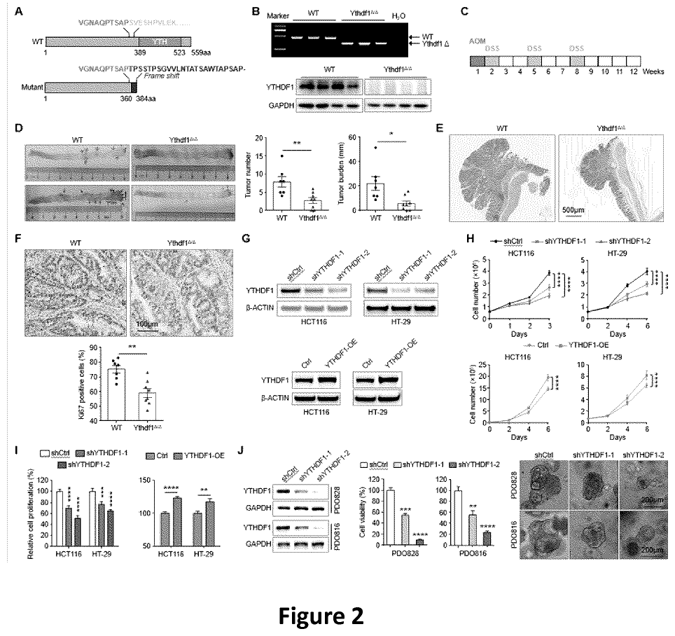

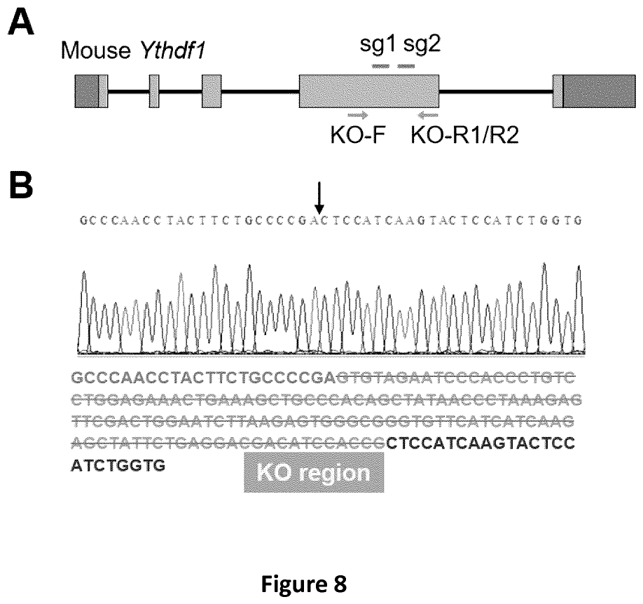

To determine the tumorigenic function of YTHDF1 in CRC, we generated Ythdf1 knockout mice by CRISPR/Cas9. CRISPR/Cas9-mediated deletion resulted in a frameshift and a premature stop codon in the exon 4 of mouse Ythdf1 gene, generating a truncated protein lacking the YTH domain and triggering nonsense-mediated mRNA decay (NMD) (FIGS. 2A and B; FIG. 8).¹⁸ Ythdf1 knockout (Ythdf1^{Δ/Δ}) and wildtype mice were given a single dose of the carcinogen azoxymethane (AOM) plus three cycles of dextran sulfate sodium (DSS) to induce colon cancer (FIG. 2C).¹⁹ Ythdf1^{Δ/Δ} mice showed a significant reduction in tumor number (P<0.01) and burden (P<05; the sum of all tumor size per mouse) compared to wildtype mice (FIGS. 2D and E).²⁰ IHC of the tumor proliferation index marker Ki-67 showed that the percentage of tumor cells with Ki-67-positive nuclear immunostaining was significantly decreased in Ythdf1-null colon tumors compared with wildtype colon tumors (FIG. 2F), inferring lower proliferation rate of Ythdf1-null CRC tumor cells. Together, these data suggest that YTHDF1 functions as an oncogenic factor in colorectal tumorigenesis.

### YTHDF1 Promotes Cell Proliferation and Metastasis Capacity of Human CRC Cells and Growth of Human CRC Organoids

We next determined the function of YTHDF1 in human CRC cell lines and patient-derived CRC organoids. YTHDF1 knockdown suppressed cell growth of HCT116 and HT-29 in vitro (FIG. 2G-I). Reciprocally, YTHDF1 overexpression significantly promoted cell proliferation (FIG. 2G-I). Consistent with the observation in CRC cell lines, YTHDF1 knockdown resulted in reduced organoids with disrupted structure grown from single CRC organoid cells and decreased cell growth in two patient-derived CRC organoids 3D culture (PDO828 and PDO816; FIG. 2J).

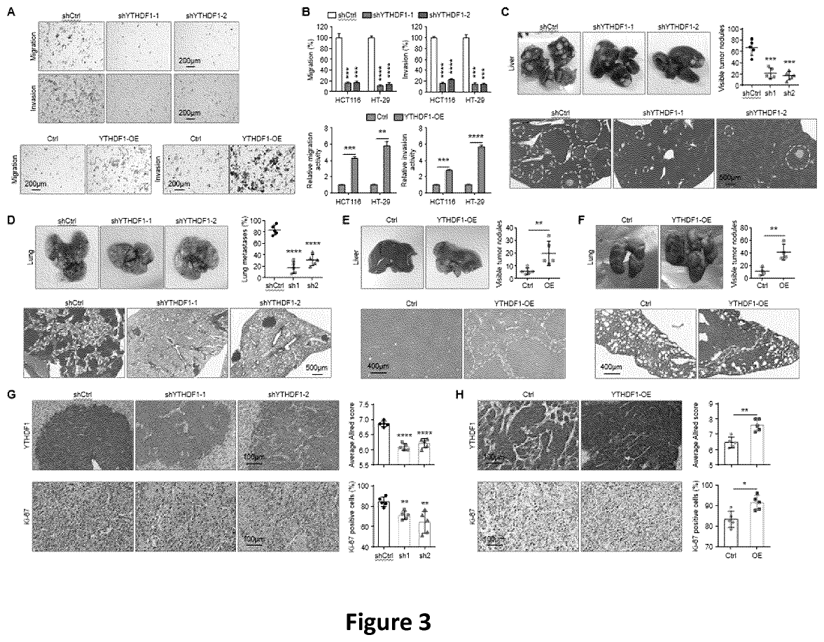

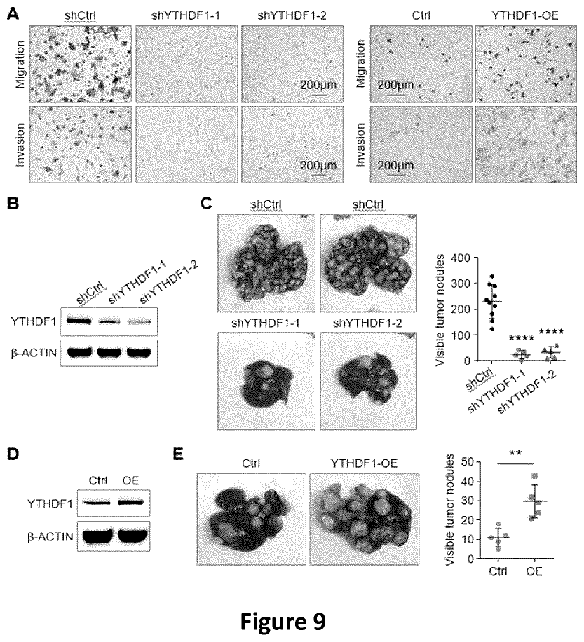

We next investigated the role of YTHDF1 in the pro-metastatic capacity of CRC cells. Transwell assay showed that YTHDF1 knockdown evidently decreased CRC cell migration and invasion in vitro, whereas cell migration and invasion abilities of CRC cells were significantly increased upon YTHDF1 overexpression (FIGS. 3A and B; FIG. 9A). YTHDF1 knockdown and overexpression assays in mouse liver and lung metastases models further confirmed the metastasis-promoting function of YTHDF1 in vivo (FIG. 3C-F; FIG. 9B-E). Besides, Ki-67 IHC showed that the liver metastases had significantly lower proportion of proliferating tumor cells upon YTHDF1 knockdown (FIG. 3G), while significantly higher proliferation rate was observed in YTHDF1-overexpressing liver metastases compared to control (FIG. 3H). Taken together, these data suggest that YTHDF1 promotes CRC cell motility, invasiveness and metastasis.

### Transcriptome Profiling Implicates the Functional Role of YTHDF1 in Tumor Growth and Metastasis

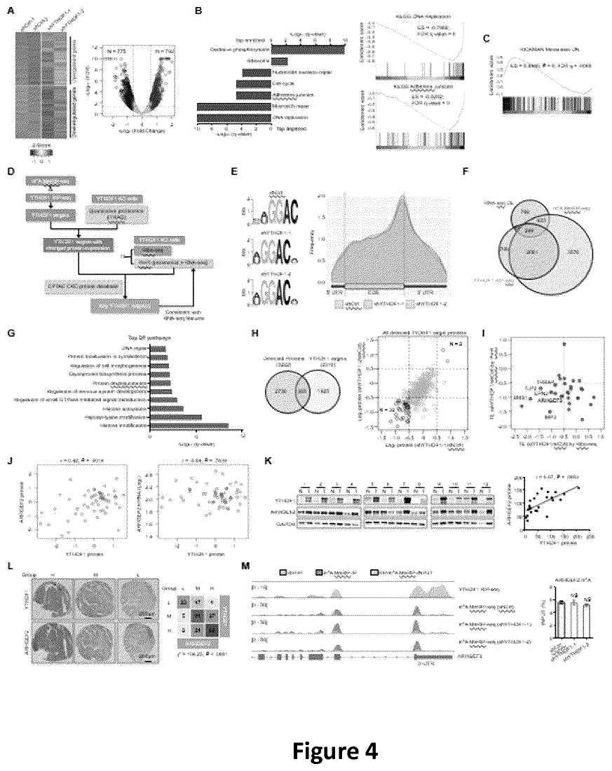

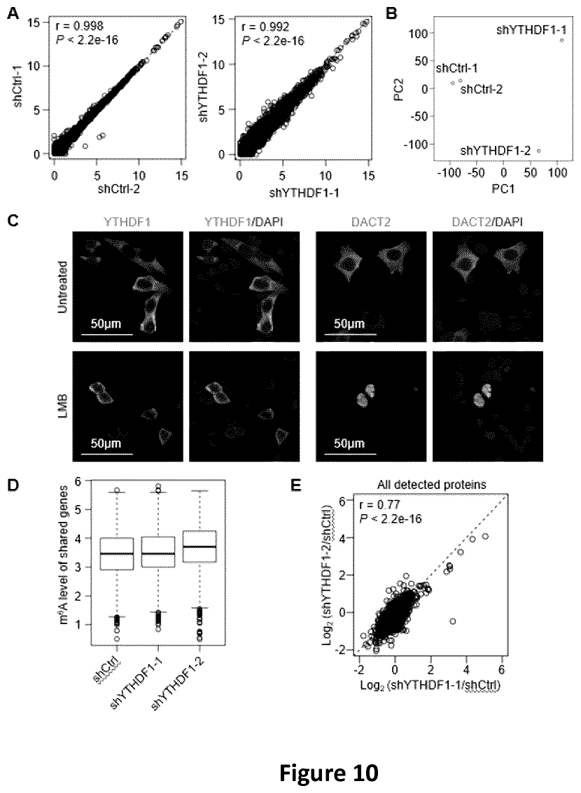

To elucidate the molecular mechanism underlying the oncogenic role of YTHDF1, we performed RNA-sequencing (RNA-seq) in YTHDF1-knockdown and control HCT116 cells (FIGS. 10A and B). Differential gene analysis using DESeq identified 792 up-regulated and 775 down-regulated genes in HCT116 cells with YTHDF1 knockdown as compared to control cells (FIG. 4A; and data not shown). GSEA analysis revealed that the top Kyoto Encyclopedia of Genes and Genomes (KEGG) gene sets depleted upon YTHDF1 knockdown were those implicated in ‘DNA replication’, ‘mismatch repair’, ‘adherens junction’ and ‘cell cycle pathways’ (FIG. 4B). Enrichment of ‘DNA replication’ and ‘cell cycle’ pathways were consistent with reduced cell survival by YTHDF1 knockdown, while ‘adherens junction’ pathway matched with the reduced cell migration and invasion capabilities of YTHDF1-knockdown cells. In addition, ‘RICKMAN Metastasis DN’ signature was significantly enriched among gene sets up-regulated in YTHDF1-knockdown cells (FIG. 4C), in accordance with GSEA result in the TCGA CRC cohort (FIG. 1E; FIG. 7E).

### Multiomic Analysis Unveils Key YTHDF1 m⁶A Targets Involved in Colorectal Tumorigenesis and Metastasis

m⁶A deposition influences RNA processing in the nucleus (e.g. splicing and export), and RNA decay and translation in the cytoplasm.^{21,22} YTHDF1 is a m⁶A reader that binds to the m⁶A-modified mRNA via YTH domain to facilitate translation in cytoplasm in Hela cells¹². We thus examined subcellular localization of YTHDF1 by immunofluorescence (IF) staining in CRC cells. YTHDF1 is localized in cytoplasm in both untreated and nuclear export inhibitor Leptomycin B (LMB)-treated HCT116 cells (FIG. 10C), indicating that YTHDF1 mainly functions in cytoplasm. To decipher how YTHDF1 interprets m⁶A modifications and modulates mRNA translation in CRC, we applied multiomics profiling to pinpoint downstream targets and pathways regulated by YTHDF1-m⁶A in CRC (FIG. 4D): 1) to define YTHDF1 m⁶A targets by m⁶A methylated RNA immunoprecipitation sequencing (m⁶A MeRIP-seq) and YTHDF1 RNA immunoprecipitation (RIP)-seq; 2) to identify YTHDF1 targets with significant changes in protein levels upon YTHDF1 knockdown by quantitative proteomics; 3) to determine the effect of YTHDF1 on translation efficiency of its targets by ribosome profiling (Ribo-seq); and 4) to verify correlation between YTHDF1 and its target gene protein abundances in CRC clinical samples.

m⁶A MeRIP-seq showed that “GGAC” was the most enriched motif found in m⁶A peaks identified from both YTHDF1-knockdown and control HCT116 cells (FIG. 4E), consistent with previous reports.⁷ We also observed a similar consistent density distribution of m⁶A peaks, i.e. enriched near stop codon, in YTHDF1-knockdown and control cells (FIG. 4E), and YTHDF1 knockdown had no effect on the overall m⁶A levels (FIG. 10D). We next performed YTHDF1 RNA immunoprecipitation sequencing (RIP-seq) to map RNA transcripts bound by YTHDF1 in HCT116 cells (data not shown). RNA pulldown led to identification of 3,325 transcripts with enrichment greater than 2-fold compared to INPUT RNA (FIG. 4F). Overlap of m⁶A MeRIP-seq and YTHDF1 RIP-seq datasets identified 2,310 YTHDF1 target genes with m⁶A modification (FIG. 4F). Functional enrichment analysis of YTHDF1 targets revealed distinct gene clusters (FIG. 4G). Among the top enriched Gene Ontology/Biological Process (GO/BP) pathways, ‘regulation of small GTPase mediated signal transduction’, ‘regulation of cell morphogenesis’, and ‘protein localization to cytoskeleton’ are directly related to function of the cytoskeleton and cell motility, which are closely associated with cell migration and invasion.²³ We also observed that mRNA levels of most YTHDF1 targets (2,061/2,310) were not significantly altered upon YTHDF1 knockdown (FIG. 4F).

ITRAQ (isobaric tagging for relative and absolute quantification)-based proteomic profiling of YTHDF1-knockdown and control HCT116 cells were performed to identify differentially expressed proteins.²⁴ Three experimental replications were performed for each group. 3,222 proteins were detected in at least two out of three replicates per group in all shYTHDF1 and control groups, and 214 differentially expressed proteins were identified in both shYTHDF1 groups compared to control (FIG. 4H; FIG. 10E; and data not shown). We integrated our proteomic dataset with YTHDF1 targets, revealing 34 genes with significant alterations in protein abundance after YTHDF1 knockdown (32 down-regulated; 2 up-regulated) (FIG. 4H; Table A). Given that YTHDF1 modulates translation, we next determined the effect of YTHDF1 knockdown on translation efficiency of the 34 genes using 1) ratio of protein-to-mRNA (PmR) from HCT116 proteomics and RNA-seq datasets; and 2) publicly available Ribo-seq data.¹² Nine genes showed both significantly decreased PmR and reduced Ribo-seq-derived translation efficiency upon knockdown of YTHDF1 (FIG. 4I). Finally, we corroborated our data in the Clinical Proteomic Tumor Analysis Consortium (CPTAC) CRC cohort to further narrow down targets whose protein abundances are positively correlated with that of YTHDF1 in human CRC (Table A).²⁵ This led to the identification of ARHGEF2, BMS1, EPN2, IMP3, TJP2 and TRRAP as key YTHDF1 targets in CRC.

TABLE A
List of YTHDF1 direct targets from multiomics analysis.

| | PmR | Ribo-seq | CPTAC | CPTAC | |
|---|---|---|---|---|---|
| | (Y1 KD/ | (Y1 KD/ | CRC protein | Spearman's | CPTAC |
| Gene | shCtrl) | siCtrl) | database | Correlation | q-value |
| ARHGEF2 | −0.872618404 | −0.850 | Positive | 0.329 | 0.0259 |
| BMS1 | −1.063319033 | −1.859724853 | Positive | 0.686 | 1.94E−05 |
| COG1 | −0.835875398 | 0.354687832 | Positive | 0.308 | 0.0313 |
| EPN2 | −0.830241907 | −1.016330128 | Positive | 0.746 | 3.18E−03 |
| HDHD3 | −1.443281855 | −0.30087297 | Positive | 0.384 | 5.23E−03 |
| IMP3 | −1.512789455 | −1.071349336 | Positive | 0.415 | 3.09E−03 |
| NPLOC4 | 0.86084686 | −0.487375718 | Positive | 0.395 | 4.63E−03 |
| TRRAP | −0.574717396 | −0.852439539 | Positive | 0.4 | 0.0203 |
| ZC3H4 | −1.266353547 | 0.587991617 | Positive | 0.444 | 3.50E−03 |
| TJP2 | −0.690794857 | −1.454249261 | Positive | 0.352 | 0.0104 |
| ZW10 | −0.426101762 | 0.129469002 | Positive | 0.334 | 0.0262 |
| CSTF1 | −0.246103047 | −0.701933053 | Positive | 0.615 | 7.45E−05 |
| DDI2 | −0.284392079 | No data | Positive | 0.449 | 7.71E−04 |
| PCYOX1 | −0.35086333 | −0.482912008 | Positive | 0.459 | 5.73E−04 |
| RIF1 | −0.361566487 | −0.660527429 | Positive | 0.314 | 0.0578 |
| RTKN | −1.230565215 | −0.279403774 | Positive | 0.366 | 0.0358 |
| SEC24B | −0.426956005 | −0.122232157 | Positive | 0.481 | 3.16E−04 |
| BRD1 | −0.717343628 | −0.221186897 | No correlation | 0.214 | 0.665 |
| URB1 | −0.740378374 | 0.244994075 | No correlation | 0.144 | 0.411 |
| PSME4 | −0.975484358 | −0.615251084 | No correlation | 0.2 | 0.408 |
| EARS2 | −0.701528582 | 0.200584153 | No correlation | 0.22 | 0.348 |
| AKAP13 | −0.530856808 | −0.465280064 | No correlation | 0.345 | 0.096 |
| GID8 | −1.23217002 | No data | No correlation | −0.571 | 0.183 |
| NR2F1 | −1.010244125 | 0.710569709 | No data | | |
| ELMO2 | −1.034736754 | 0.069674944 | No data | | |
| FOXK2 | −0.638565621 | −0.22134916 | No data | | |
| NCOA3 | −0.86647747 | −0.322102486 | No data | | |
| PER2 | 1.351885307 | No data | No data | | |
| TFIP11 | 1.041049438 | 0.060707828 | No data | | |
| ZFP36L2 | 1.323894506 | −2.218458853 | No data | | |
| WNT16 | −0.485720488 | No data | No data | | |
| ROR2 | −0.972773674 | −0.569818654 | No data | | |
| ZNF574 | −1.037886758 | −0.326104124 | No data | | |
| WDR6 | −1.795756 | 0.244475146 | No data | | |

### ARHGEF2 is a Critical Target of YTHDF1 in Multiple CRC Models

Amongst the 6 key YTHDF1 targets, ARHGEF2 belongs to the regulators of Rho family of GTPases, one of the top pathways enriched in YTHDF1 targets (FIG. 4G).²⁶ In the CPTAC CRC cohort, ARHGEF2 protein, but not its mRNA, was positively correlated with YTHDF1 protein (FIG. 4J). The correlation between ARHGEF2 and YTHDF1 protein abundances was further confirmed in 12 pairs of matched CRC tumor and non-tumor tissue samples by Western blot (r=0.67; P=0.0003; FIG. 4K) and a tissue microarray analysis containing 208 human CRC tissues (X²=104.23; P<0001; FIG. 4L). Furthermore, both ARHGEF2 and YTHDF1 protein levels were significantly associated with metastasis (P=0.035 and 0.007) and advanced TNM stage (P=0.049 and 0.012) in the tissue microarray dataset (data not shown).

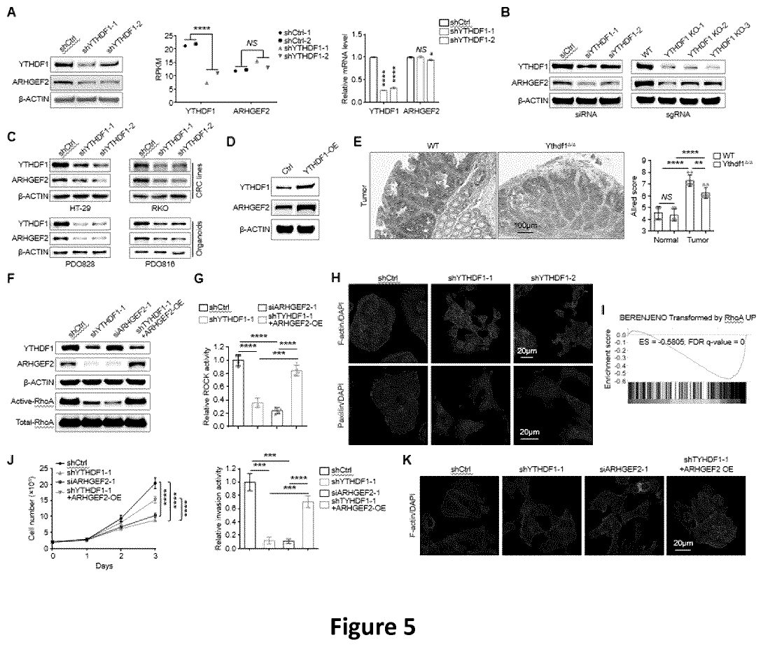

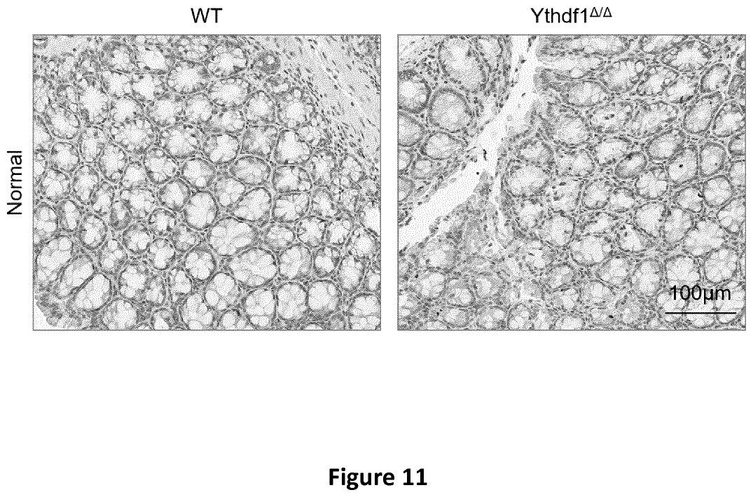

Binding of YTHDF1 to ARHGEF2 overlapped with m⁶A peaks in the 3′-untranslated region (UTR) near the stop codon of ARHGEF2 mRNA (FIG. 4M). m⁶A MeRIP PCR showed that m⁶A levels near the stop codon of ARHGEF2 mRNA was not significantly altered upon YTHDF1 knockdown (FIG. 4M), indicating that attenuated protein translation of ARHGEF2 was not due to changes in m⁶A modification but reduced YTHDF1 abundance. Western blot, RNA-seq and qPCR confirmed that YTHDF1 knockdown inhibited ARHGEF2 protein expression with negligible changes in its mRNA levels in HCT116 cells (FIG. 5A). To rule out the off-target effects by shRNA, down-regulation of ARHGEF2 protein expression was validated by use of YTHDF1 siRNAs and sgRNAs (FIG. 5B). Furthermore, ARHGEF2 protein expression was evidently decreased upon YTHDF1 knockdown in two additional CRC cell lines HT-29 and RKO, as well as in two patient-derived CRC organoid PDO828 and PDO816 (FIG. 5C). On the contrary, overexpression of YTHDF1 induced ARHGEF2 protein levels (FIG. 5D). In AOM/DSS-induced CRC in mice, ARHGEF2 protein was induced in colon tumors compared with normal colon tissues, whereas Ythdf1 knockout led to a significant reduction in ARHGEF2 protein expression in mouse colon tumors compared with wildtype group (FIG. 5E; FIG. 11). Taken together, our data suggests that YTHDF1 promotes translation of ARHGEF2 in CRC cell lines, organoids, genetic mouse models and human CRC patients.

### ARHGEF2 is a Critical Target for YTHDF1-Mediated Oncogenic Transformation

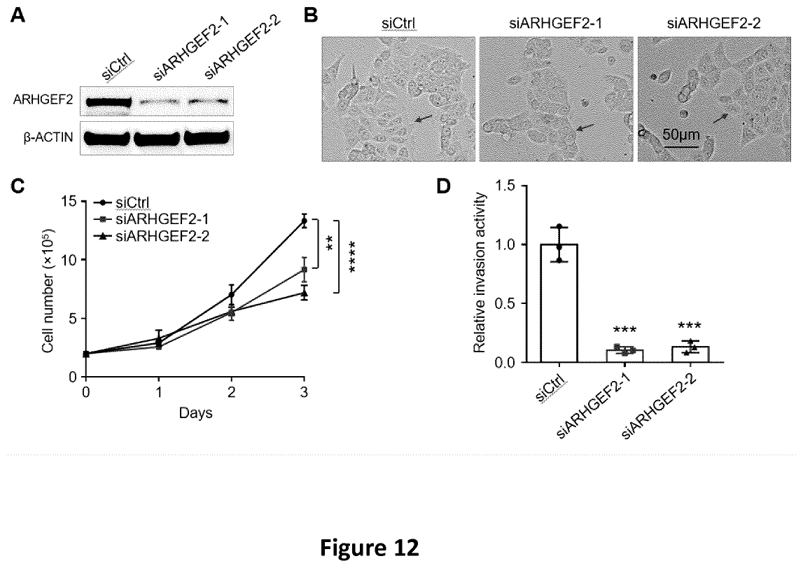

ARHGEF2 functions as a RhoA-specific guanine nucleotide exchange factor (GEF) for Rho GTPase, activating RhoA signaling to promote the induction of stress fibers and focal adhesions.²⁷⁻³⁰ Consistently, YTHDF1-knockdown cells displayed lowered levels of active RhoA (FIG. 5F), reduced activity of RhoA downstream effector Rho-associated protein kinase (ROCK) (FIG. 5G), dampened stress fibre formation and focal adhesion (FIG. 5H). Moreover, GSEA showed depletion of constitutively active RhoA-induced gene signature (BERENJENO Transformed by RhoA UP) in YTHDF1-knockdown cells (FDR q-value=0; FIG. 5I). Reciprocally, ARHGEF2 silencing phenocopied YTHDF1 loss by inducing changes in cell morphology, and inhibiting cell viability and invasion in CRC cells (FIG. 12).

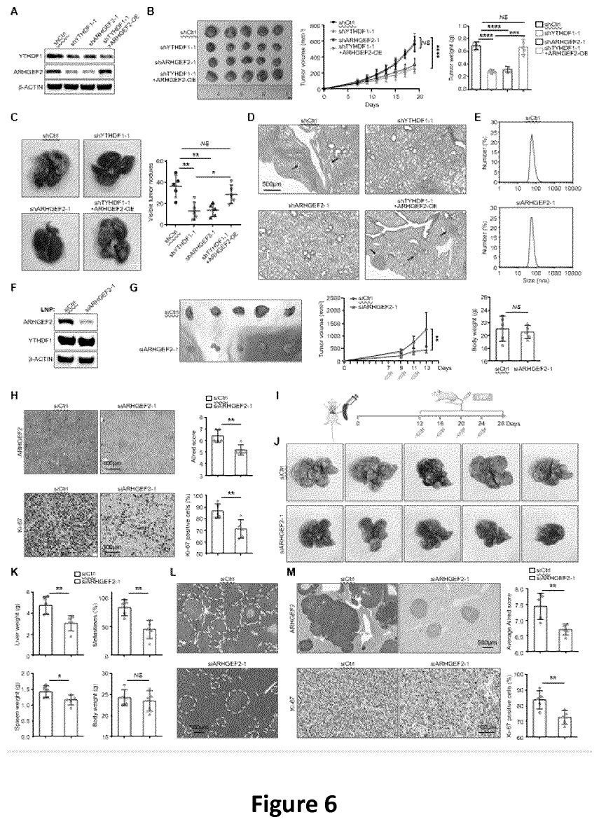

Rescue assays were performed to confirm whether ARHGEF2 is indispensable for YTHDF1-induced oncogenic transformation. ARHGEF2 re-expression in YTHDF1-knockdown HCT116 cells significantly restored cell proliferation and invasion in vitro (FIG. 5J). In addition, ARHGEF2 is involved in YTHDF1-mediated RhoA signaling in CRC cells, as its re-expression rescued active RhoA expression (FIG. 5F), ROCK activity (FIG. 5G) and stress fibre formation (FIG. 5K) in YTHDF1-knockdown cells. Furthermore, ARHGEF2 fully rescued the growth of HCT116 xenografts and lung metastasis upon knockdown of YTHDF1 (FIG. 6A-D).

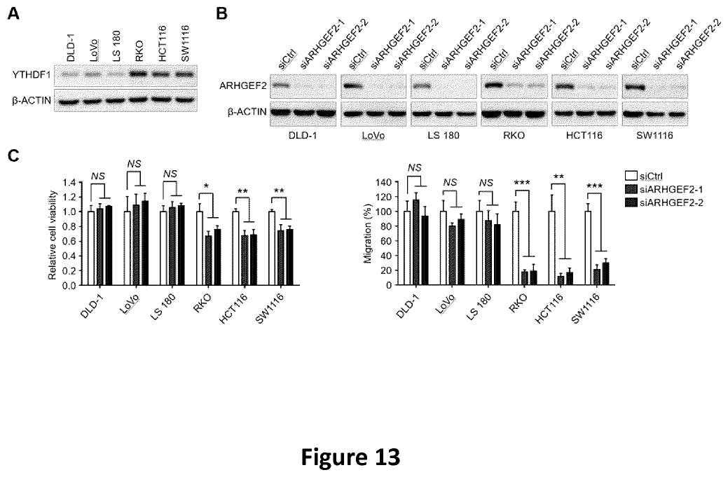

Since our data demonstrated that ARHGEF2 is a key functional target of YTHDF1, we next asked if targeting ARHGEF2 was more effective in CRC cells with high YTHDF1 expression. ARHGEF2 knockdown led to significantly decreased cell growth and migration abilities in YTHDF1-high CRC cell lines (HCT116, RKO and SW1116) but not in YTHDF1-low CRC cell lines (DLD-1, LoVo and LS 180) (FIG. 13), suggesting that CRC tumors with YTHDF1 high expression are more sensitive to ARHGEF2 inhibition.

Targeting ARHGEF2 Using LNP siRNA for CRC Therapy

siRNA nanoparticles with improved biocompatibility have shown attractive potential for disease treatments as theoretically any gene could be targeted by this approach.³¹ Since no drug is available to directly target ARHGEF2, we used a Food and Drug Administration (FDA) approved LNP formulation technology for siRNA delivery to target ARHGEF2.³² To improve in vivo stability, all pyrimidine bases (C/U) in both strands of siRNAs were modified by 2′-O-Methyl (2′-OMe).³³ siRNA encapsulation efficiency (siCtrl: 91.6%; siARHGEF2-1:93.7%) and the hydrodynamic size (siCtrl: 65.38±0.50 nm, Polydispersity Index (PDI): 0.112; siARHGEF2-1:60.45±2.35 nm, PDI: 0.05) and dispersity of LNP (FIG. 6E) were confirmed before knockdown assays. In vitro knockdown assays showed that LNP 2′-OMe-modified siARHGEF2-1 efficiently knocked down ARHGEF2 gene expression at 100 nM LNP-siRNA concentration (FIG. 6F).

It has been reported that LNP siRNA is well distributed throughout the subcutaneous xenograft hours after intratumoral injection and the knockdown effect of a single dose could last for up to 4 days.³⁴ Therefore, in our xenograft model, after subcutaneous tumor volume reached approximately 100 mm³, the mice were randomly assigned for treatment with LNP siCtrl or LNP siARHGEF2-1 (2 mg/kg on day7, day9, day11) every two days via intratumoral injection for 7 days. Consistent with in vitro experiments (FIG. 12C), LNP siARHGEF2-1 significantly inhibited the growth of HCT116 xenografts compared to the control group (P<0.01) with negligible changes in mice body weight (FIG. 6G). IHC of ARHGEF2 and Ki-67 confirmed reduced expression of ARHGEF2 and slowed growth rate in LNP siARHGEF2-1-treated tumors compared to the control group (FIG. 6H).

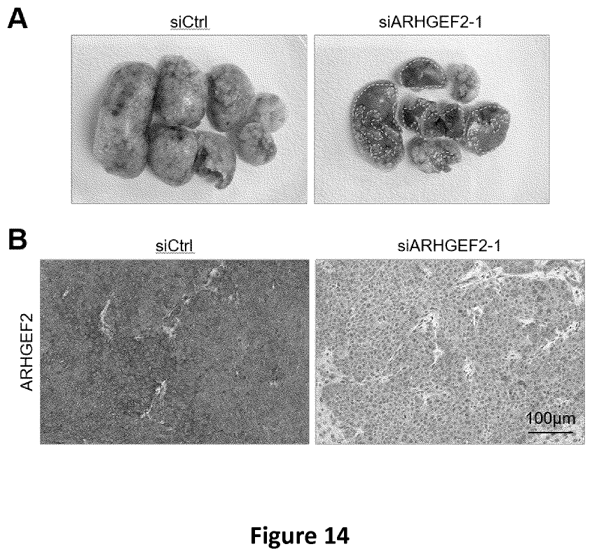

Liver is the most common site of metastasis for CRC patients. Over 50% of CRC patients develop liver metastases over the course of their lives, and approximately 14%˜18% of CRC patients have liver metastases at their initial medical consultation.^{35,36} Reducing liver metastases would be of great importance for CRC patients. Thus, the high accumulation of Onpattro LNP in the liver prompted us to test the potential of LNP siARHGEF2-1 for inhibiting liver metastases in vivo following intravenous administration.³² The mouse liver metastasis model was established by injecting 7.5×10⁵ HCT116 cells intrasplenically into each NOD/SCID mouse. Twelve days after surgery, the mice were randomly assigned for treatment with LNP siCtrl or LNP siARHGEF2-1 every four days (2 mg/kg on day12, day16, day20, day24) via tail-vein injection for 16 days (FIG. 6I). Our data showed that LNP siARHGEF2-1 significantly reduced colorectal liver metastases compared to the LNP siCtrl-treated group as evidenced by significantly reduced liver weight (P<01) and less proportion of tumor metastases in the mouse livers (P<0.01; FIG. 6J-L; FIG. 14A). LNP siARHGEF2-1 treatment via tail-vein injection led to significant reduction in ARHGEF2 expression and decreased proportion of Ki-67-positive proliferating tumor cells in liver metastases (FIG. 6M; FIG. 14B). Taken together, our data suggests targeting ARHGEF2 is a promising therapeutic strategy for CRC.

Emerging evidence implies crucial roles of m⁶A epitranscriptome in every hallmark of cancer biology.⁹ In this study, we identified YTHDF1 as the most highly up-regulated m⁶A regulator in CRC, implying its role in converting deregulated m⁶A modifications to pro-tumorigenic signals. Noticeably, YTHDF1 expression in human CRC is correlated with metastatic progression. Using Ythdf1 knockout mice, CRC cell lines and primary CRC organoids, we demonstrated that YTHDF1 exerts pro-tumorigenic effects by enhancing tumor growth, migration/invasion and metastasis.

YTHDF1 is a cytoplasmic m⁶A reader that affects protein translation via interacting with translation machinery.¹² In this regard, multiomics integration analysis was performed to characterize key YTHDF1 target(s) responsible for its pro-tumorigenic functions. We pinpointed ARHGEF2 for further investigation. We validated YTHDF1-m⁶A-ARHGEF2 axis as a critical molecular signaling involved in YTHDF1-mediated tumorigenesis and metastasis in vitro and in vivo. ARHGEF2 functions to activate RhoA signaling as a RhoA-specific GEF for Rho GTPase, and it mainly participates in cytoskeleton dynamics, focal adhesion and stress fibre formation under physiological conditions.^{28,37} In support of our data, ARHGEF2 has been reported to promote cell growth and survival by regulating cell cycle transition, apoptosis and transformation.^{27,38} Pro-tumorigenic effects of ARHGEF2 involved both RhoA-dependent and -independent mechanisms, such as induction of RAS/MAPK.³⁸

Recent studies have uncovered alternative targets of YTHDF1 in human cancers. For example, YTHDF1 has been shown to modulate the canonical WNT/β-catenin signaling through affecting the translation efficiency of upstream WNT regulators FZD9 and WNT6 in human CRC cells, as well as β-catenin transcriptional partner TCF7L2 in mice intestines.^{39,40} However, those proteins were not detected in our proteomic dataset or the CPTAC CRC cohort, which may be due to low expression of those proteins in CRC. Although it is likely that YTHDF1 regulates multiple signal pathways, the fact that ARHGEF2 overexpression can rescue gene expression and phenotypes of YTHDF1 knockdown suggests it is a main target.

MSI-H/dMMR CRC subtype accounts for only 5% of metastatic CRC.⁴¹ YTHDF1 is mainly amplified in non-MSI-H/dMMR CRC patients (data not shown), which comprise 95% of metastatic CRC cases and don't respond to immunotherapy and have limited options for targeted therapy.⁴² Therefore, targeting the YTHDF1-m⁶A-ARHGEF2 axis might be a promising therapeutic approach. The recent key advances in LNP delivery technology with efficient encapsulation of siRNA and favorable pharmacokinetics and safety highlighted the potential of developing LNP siRNA drugs targeting YTHDF1-m⁶A-ARHGEF2 axis.³² The LNP siARHGEF2 system was shown to have an efficient targeting function in vitro and its potency was validated in the xenograft and liver metastasis model. Collectively, the LNP siARHGEF2 drug may provide a potential therapeutic option for metastatic CRC patients through modulation of the epitranscriptome.

In summary, m⁶A reader YTHDF1 promotes CRC tumorigenesis and metastasis through up-regulation of ARHGEF2 translation and protein expression. Given the widespread YTHDF1 upregulation in CRC and its pro-oncogenic role in CRC pathogenesis, targeting YTHDF1-m⁶A-ARHGEF2 axis could be a promising therapeutic strategy for inhibition of CRC progression and metastasis. Finally, our work highlights the potential of LNP siARHGEF2 drug for tumor treatment.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Siegel R L, Miller K D, Goding Sauer A, et al. Colorectal cancer statistics, 2020. CA Cancer J Clin 2020; 70:145-164.
2. Davis L E. The evolution of biomarkers to guide the treatment of metastatic colorectal cancer. Am J Manag Care 2018; 24: S107-S117.
3. Engstrand J, Nilsson H, Stromberg C, et al. Colorectal cancer liver metastases—a population-based study on incidence, management and survival. BMC Cancer 2018; 18. Available at: http://dx.doi.org/10.1186/s12885-017-3925-x.
4. International Cancer Genome Consortium, Hudson T J, Anderson W, et al. International network of cancer genome projects. Nature 2010; 464:993-998.
5. Cancer Genome Atlas Network. Comprehensive molecular characterization of human colon and rectal cancer. Nature 2012; 487:330-337.
6. Peer E, Rechavi G, Dominissini D. Epitranscriptomics: regulation of mRNA metabolism through modifications. Curr Opin Chem Biol 2017; 41:93-98.
7. Dominissini D, Moshitch-Moshkovitz S, Schwartz S, et al. Topology of the human and mouse m⁶A RNA methylomes revealed by m⁶A-seq. Nature 2012; 485:201-206.
8. Zeng Y, Wang S, Gao S, et al. Refined RIP-seq protocol for epitranscriptome analysis with low input materials. PLoS Biol 2018; 16: e2006092.
9. Huang H, Weng H, Chen J. mA Modification in Coding and Non-coding RNAs: Roles and Therapeutic Implications in Cancer. Cancer Cell 2020; 37:270-288.
10. Shi H, Wei J, He C. Where, When, and How: Context-Dependent Functions of RNA Methylation Writers, Readers, and Erasers. Mol Cell 2019; 74:640-650.
11. Zhou K I, Pan T. An additional class of mA readers. Nat Cell Biol 2018; 20:230-232.
12. Wang X, Zhao B S, Roundtree I A, et al. N (6)-methyladenosine Modulates Messenger RNA Translation Efficiency. Cell 2015; 161:1388-1399.
13. Shi H, Zhang X, Weng Y L, et al. mA facilitates hippocampus-dependent learning and memory through YTHDF1. Nature 2018; 563:249-253.
14. Han D, Liu J, Chen C, et al. Anti-tumour immunity controlled through mRNA mA methylation and YTHDF1 in dendritic cells. Nature 2019; 566:270-274.
15. Zhuang M, Li X, Zhu J, et al. The m⁶A reader YTHDF1 regulates axon guidance through translational control of Robo3.1 expression. Nucleic Acids Res 2019; 47:4765-4777.
16. Wang S, Huang J, Li C, et al. MAP9 Loss Triggers Chromosomal Instability, Initiates Colorectal Tumorigenesis, and Is Associated with Poor Survival of Patients with Colorectal Cancer. Clin Cancer Res 2020; 26:746-757.
17. Mermel C H, Schumacher S E, Hill B, et al. GISTIC2.0 facilitates sensitive and confident localization of the targets of focal somatic copy-number alteration in human cancers. Genome Biol 2011; 12: R41.
18. Popp M W, Maquat L E. Leveraging Rules of Nonsense-Mediated mRNA Decay for Genome Engineering and Personalized Medicine. Cell 2016; 165:1319-1322.
19. Fazio V, Robertis M, Massi E, et al. The AOM/DSS murine model for the study of colon carcinogenesis: From pathways to diagnosis and therapy studies. Journal of Carcinogenesis 2011; 10:9. Available at: http://dx.doi.org/10.4103/1477-3163.78279.
20. Neufert C, Becker C, Neurath M F. An inducible mouse model of colon carcinogenesis for the analysis of sporadic and inflammation-driven tumor progression. Nat Protoc 2007; 2:1998-2004.
21. Huang H, Weng H, Zhou K, et al. Histone H3 trimethylation at lysine 36 guides mA RNA modification co-transcriptionally. Nature 2019; 567:414-419.
22. Yang Y, Hsu P J, Chen Y S, et al. Dynamic transcriptomic m⁶A decoration: writers, erasers, readers and functions in RNA metabolism. Cell Research 2018; 28:616-624. Available at: http://dx.doi.org/10.1038/s41422-018-0040-8.
23. Fife C M, McCarroll J A, Kavallaris M. Movers and shakers: cell cytoskeleton in cancer metastasis. Br J Pharmacol 2014; 171:5507-5523.
24. Wiese S, Reidegeld K A, Meyer H E, et al. Protein labeling by iTRAQ: a new tool for quantitative mass spectrometry in proteome research. Proteomics 2007; 7:340-350.
25. Vasaikar S, Huang C, Wang X, et al. Proteogenomic Analysis of Human Colon Cancer Reveals New Therapeutic Opportunities. Cell 2019; 177:1035-1049.e19.
26. Sadok A, Marshall C J. Rho GTPases: masters of cell migration. Small G TPases 2014; 5: e29710.
27. Birkenfeld J, Nalbant P, Yoon S H, et al. Cellular functions of GEF-H1, a microtubule-regulated Rho-GEF: is altered GEF-H1 activity a crucial determinant of disease pathogenesis? Trends Cell Biol 2008; 18:210-219.
28. Huang I H, Hsiao C T, Wu J C, et al. GEF-H1 controls focal adhesion signaling that regulates mesenchymal stem cell lineage commitment. J Cell Sci 2014; 127:4186-4200.
29. Sandi M J, Marshall C B, Balan M, et al. MARK3-mediated phosphorylation of ARHGEF2 couples microtubules to the actin cytoskeleton to establish cell polarity. Sci Signal 2017; 10. Available at: http://dx.doi.org/10.1126/scisignal.aan3286.
30. Cao J, Yang T, Tang D, et al. Increased expression of GEF-H1 promotes colon cancer progression by RhoA signaling. Pathol Res Pract 2019; 215:1012-1019.
31. Kulkarni J A, Cullis P R, Meel R van der. Lipid Nanoparticles Enabling Gene Therapies: From Concepts to Clinical Utility. Nucleic Acid Ther 2018; 28:146-157.
32. Akinc A, Maier M A, Manoharan M, et al. The Onpattro story and the clinical translation of nanomedicines containing nucleic acid-based drugs. Nat Nanotechnol 2019; 14:1084-1087.
33. Kenski D M, Butora G, Willingham A T, et al. siRNA-optimized Modifications for Enhanced In Vivo Activity. Mol Ther Nucleic Acids 2012; 1: e5.
34. Yanagi T, Tachikawa K, Wilkie-Grantham R, et al. Lipid Nanoparticle-mediated siRNA Transfer Against PCTAIRE1/PCTK1/Cdk16 Inhibits In Vivo Cancer Growth. Mol Ther Nucleic Acids 2016; 5: e327.
35. Zarour L R, Anand S, Billingsley K G, et al. Colorectal Cancer Liver Metastasis: Evolving Paradigms and Future Directions. Cell Mol Gastroenterol Hepatol 2017; 3:163-173.
36. Jegatheeswaran S, Mason J M, Hancock H C, et al. The liver-first approach to the management of colorectal cancer with synchronous hepatic metastases: a systematic review. JAMA Surg 2013; 148:385-391.
37. Pathak R, Dermardirossian C. GEF-H1: orchestrating the interplay between cytoskeleton and vesicle trafficking. Small GTPases 2013; 4:174-179.
38. Cullis J, Meiri D, Sandi M J, et al. The RhoGEF GEF-H1 is required for oncogenic RAS signaling via KSR-1. Cancer Cell 2014; 25:181-195.
39. Bai Y, Yang C, Wu R, et al. YTHDF1 Regulates Tumorigenicity and Cancer Stem Cell-Like Activity in Human Colorectal Carcinoma. Front Oncol 2019; 9:332.
40. Han B, Yan S, Wei S, et al. YTHDF1-mediated translation amplifies Wnt-driven intestinal stemness. EMBO Rep 2020; 21: e49229.
41. Battaglin F, Naseem M, Lenz H J, et al. Microsatellite instability in colorectal cancer: overview of its clinical significance and novel perspectives. Clin Adv Hematol Oncol 2018; 16:735-745.
42. Xie Y H, Chen Y X, Fang J Y. Comprehensive review of targeted therapy for colorectal cancer. Signal Transduct Target Ther 2020; 5:22.

### Methods References

m1. Pastuła A, Middelhoff M, Brandtner A, et al. Three-Dimensional Gastrointestinal Organoid Culture in Combination with Nerves or Fibroblasts: A Method to Characterize the Gastrointestinal Stem Cell Niche. Stem Cells Int 2016; 2016:3710836.
m2. Wang S, Dong Y, Zhang Y, et al. DACT2 is a functional tumor suppressor through inhibiting Wnt/β-catenin pathway and associated with poor survival in colon cancer. Oncogene 2015; 34:2575-2585.
m3. Zeng Y, Wang S, Gao S, et al. Refined RIP-seq protocol for epitranscriptome analysis with low input materials. PLoS Biol 2018; 16: e2006092.
m4. Dobin A, Davis C A, Schlesinger F, et al. STAR: ultrafast universal RNA-seq aligner. Bioinformatics 2013; 29:15-21.
m5. Harrow J, Frankish A, Gonzalez J M, et al. GENCODE: the reference human genome annotation for The ENCODE Project. Genome Res 2012; 22:1760-1774.
m6. Love M I, Huber W, Anders S. Moderated estimation of fold change and dispersion for RNA-seq data with DESeq2. Genome Biol 2014; 15:550.
m7. Anders S, Pyl P T, Huber W. HTSeq—a Python framework to work with high-throughput sequencing data. Bioinformatics 2015; 31:166-169.
m8. Bailey T L. DREME: motif discovery in transcription factor ChIP-seq data. Bioinformatics 2011; 27:1653-1659.
m9. Cui X, Wei Z, Zhang L, et al. Guitar: An R/Bioconductor Package for Gene Annotation Guided Transcriptomic Analysis of RNA-Related Genomic Features. BioMed Research International 2016; 2016:1-8. Available at: http://dx.doi.org/10.1155/2016/8367534.
m10. Wang X, Zhao B S, Roundtree I A, et al. N (6)-methyladenosine Modulates Messenger RNA Translation Efficiency. Cell 2015; 161:1388-1399.
m11. Raudvere U, Kolberg L, Kuzmin I, et al. g: Profiler: a web server for functional enrichment analysis and conversions of gene lists (2019 update). Nucleic Acids Research 2019; 47: W191-W198. Available at: http://dx.doi.org/10.1093/nar/gkz369.
m12. Yu G, Wang L G, Han Y, et al. clusterProfiler: an R Package for Comparing Biological Themes Among Gene Clusters. OMICS: A Journal of Integrative Biology 2012; 16:284-287. Available at: http://dx.doi.org/10.1089/omi.2011.0118.
m13. Belliveau N M, Huft J, Lin P J, et al. Microfluidic Synthesis of Highly Potent Limit-size Lipid Nanoparticles for In Vivo Delivery of siRNA. Mol Ther Nucleic Acids 2012; 1: e37.

1. A method for the treatment of cancer in a subject in need thereof, the method comprising downregulating YTHDF1 or ARHGEF2.

2. The method of claim 1, wherein the method comprises modulating the YTHDF1-ARHGEF2 axis.

3. The method of claim 1, wherein downregulating ARHGEF2 comprises administration of a nucleic acid molecule to the subject, the nucleic acid molecule capable of selectively inhibiting, at least partially, ARHGEF2 expression.

4. The method of claim 3, wherein the nucleic acid molecule is a shRNA, siRNA, miRNA or antisense oligonucleotide targeted to ARHGEF2.

5. The method of claim 3, wherein the nucleic acid molecule is a shRNA, siRNA, miRNA or antisense oligonucleotide targeted to YTHDF1.

6. The method of claim 4, wherein the nucleic acid molecule is a siRNA.

7. The method of claim 6, wherein the SiRNA comprises sense strand GGAUCUACCUGUCACUACUtt (SEQ ID NO. 1) and antisense sense strand AGUAGUGACAGGUAGAUCCag (SEQ ID NO. 2).

8. The method of claim 4, wherein the nucleic acid molecule is a miRNA.

9. The method of claim 4, wherein the nucleic acid molecule is an antisense oligonucleotide.

10. The method of claim 4, wherein the nucleic acid molecule is a shRNA.

11. The method of claim 10, wherein the shRNA comprises shYTHDF1-1: 5′-CCCAGATGGATCTGCATTTAT-3′ (SEQ ID NO. 3); shYTHDF1-2: 5′-CGACATCCACCGCTCCATTAA-3′ (SEQ ID NO. 4); shARHGEF2-1: 5′-GTGCTATGCCTGTAACAAG-3′ (SEQ ID NO. 5); or shARHGEF2-2: 5′-GACGAAGCAGAGGTAATCT-3′ (SEQ ID NO. 6).

12. The method of claim 3, wherein the nucleic acid molecule is administered to the subject in a lipid nanoparticle as delivery vehicle.

13. The method of claim 1, wherein the cancer is selected from the group consisting of colorectal adenocarcinoma, stomach adenocarcinoma, lung adenocarcinoma, breast carcinoma, cholangiocarcinoma, liver hepatocellular carcinoma, head/neck squamous cell carcinoma, uterine corpus endometrial carcinoma, high-risk Wilms tumor, esophageal carcinoma, bladder urothelial carcinoma, kidney renal papillary cell carcinoma, prostate adenocarcinoma, giolblastoma multiforme, cervical squamous cell carcinoma/endocervical adenocarcinoma, pheochromocytoma/paraganglioma, and pancreatic adenocarcinoma.

14. The method of claim 13, wherein the cancer is colorectal adenocarcinoma.

15. A nucleic acid molecule capable of selectively inhibiting, at least partially, YTHDF1 or ARHGEF2 expression.

16. The nucleic acid molecule of claim 15, wherein the nucleic acid molecule is a siRNA or shRNA.

17. The nucleic acid molecule of claim 16, wherein the siRNA comprises sense strand GGAUCUACCUGUCACUACUtt (SEQ ID NO. 1) and antisense sense strand AGUAGUGACAGGUAGAUCCag (SEQ ID NO. 2).

18. The nucleic acid molecule of claim 16, wherein the shRNA comprises shYTHDF1-1: 5′-CCCAGATGGATCTGCATTTAT-3′ (SEQ ID NO. 3); shYTHDF1-2: 5′-CGACATCCACCGCTCCATTAA-3′ (SEQ ID NO. 4); shARHGEF2-1: 5′-GTGCTATGCCTGTAACAAG-3′ (SEQ ID NO. 5); or shARHGEF2-2: 5′-GACGAAGCAGAGGTAATCT-3′ (SEQ ID NO. 6).

19. The nucleic acid molecule of claim 18, wherein the nucleic acid molecule is a miRNA.

20. The nucleic acid molecule of claim 19, wherein the nucleic acid molecule is an antisense oligonucleotide.

21. The nucleic acid molecule of claim 15, encapsulated within a lipid nanoparticle.

22. (canceled)

23. (canceled)

24. A pharmaceutical composition comprising the nucleic acid molecule of claim 15, along with a pharmaceutically acceptable carrier.
